(19) United States
(12) Patent Application Publication
(10) Pub. No.: US 2023/0258644 A1
(43) Pub. Date: Aug. 17, 2023

(54) CAPTURE FLOW ASSAY DEVICE AND METHODS

(71) Applicants: B. G. NEGEV TECHNOLOGIES AND APPLICATIONS LTD., AT BEN-GURION UNIVERSITY, Beer Sheva (IL); RAMBAM MED-TECH LTD, Haifa (IL)

(72) Inventors: Robert MARKS, Omer (IL); Yehuda CHOWERS, Tel Aviv (IL); Tim AXELROD, Haniel (IL); Sigal PRESSMAN, Pardes Hannah (IL); Ron RAMON, Rehovot (IL)

(21) Appl. No.: 18/012,551

(22) PCT Filed: Jun. 24, 2021
(86) PCT No.: PCT/IL2021/050779

§ 371 Date: Dec. 22, 2022

Related U.S. Application Data
(60) Provisional application No. 63/043,830, filed on Jun. 25, 2020.

(51) Int. Cl. G01N 33/574; G01N 33/543; G01N 33/564

(52) U.S. Cl. CPC G01N 33/57488; G01N 33/54388; G01N 33/564

(57) ABSTRACT

Disclosed is a device including at least 4 sections with a unique layout which includes a surface functionalized with an agent having specific binding affinity to a target molecule, and which allows lateral flow. Disclosed are also a kit and a method for determining and quantifying the presence of an analyte in a sample.

### CROSS-REFERENCE TO RELATED APPLICATION

This application claims the benefit of priority of U.S. Provisional Patent Application No. 63/043,830, titled “CAPTURE FLOW ASSAY DEVICE AND METHODS”, filed Jun. 25, 2020, the contents of which are incorporated herein by reference in their entirety.

### FIELD OF THE INVENTION

The present invention is in the field of binding assay methods and apparatus for testing biological samples.

### BACKGROUND

The use of biologic drugs targeting, e.g., tumor necrosis factor alpha (TNF), immune checkpoint, etc., in the context of treating immune disorders, and cancer, respectively, is often hampered by an immunogenic response in the treated subject leading to the production of anti-drug antibodies (ADA), among them neutralizing antibodies, which reduce drug efficacy by both increasing its clearance and targeting its binding site, thereby neutralizing the therapeutic effect.

There is still a great need for means and methods for determining the production of neutralizing ADA in patients for, e.g., anti-TNF antibodies, immune checkpoint inhibitors, etc.

### SUMMARY

The present invention, in some embodiments, relates to a lateral flow device. In some embodiments, the device is a point of care testing device.

According to a first aspect, there is provided a device comprising a section 1, at least one section 2, at least one section 3, and a section 4, wherein: (a) the section 1 is coupled to the at least one section 2; and the section 3 is coupled to the section 2 and to the section 4; (b) the at least one section 3 comprises a surface functionalized with a target molecule and an agent having a specific binding affinity to the target molecule; and (c) sections 1 to 4 are: (i) arranged along a horizontal axis; and (ii) in liquid communication, allowing lateral flow of a liquid sequentially from the sections 1 to 4.

According to another aspect, there is provided method for determining the presence of an analyte in a sample, comprising the steps of: (a) contacting section 1 of the device of the invention with a sample; and (b) detecting the presence of a signal, wherein the presence of the signal is indicative of the presence of the analyte in the sample, thereby determining the presence of the analyte in the sample.

In some embodiments, (a) the section 1 comprises a sample collecting surface; and (b) the at least one section 2 comprises a surface comprising the agent and an agent probing molecule having specific binding affinity to the agent.

In some embodiments, the at least one section 2 comprises two separate sections 2, wherein a first section 2 comprises a surface comprising the agent, and a second section 2 comprises a surface comprising the agent probing molecule having specific binding affinity to the agent.

In some embodiments, the probing molecule is linked to a reporter molecule, and wherein the reporter molecule generates a trigger.

In some embodiments, the at least one section 3 comprises two separate sections 3, wherein a first section 3 comprises a surface functionalized with a target molecule, and second section 3 comprises a surface functionalized with an agent having a specific binding affinity to the target molecule.

In some embodiments, the section 4 comprises a surface in contact with a substrate molecule generating a signal in response to the trigger.

In some embodiments, the device further comprises a section 5.

In some embodiments, the section 5 comprises a surface in contact with a substrate molecule generating a signal in response to the trigger.

In some embodiments, the reporter molecule is selected from the group consisting of: an enzyme, a radioactive molecule, a luminescent compound, a fluorescent compound, a magnetic particle, an electro-chemiluminescent compound, a fluorescence transducing aptamer, and an electrochemically active compound.

In some embodiments, the trigger comprises: a reactive compound, electromagnetic radiation, a charged particle, or any combination thereof

In some embodiments, the section 3 and the section 4 are devoid of the probing molecule and the reporter molecule.

In some embodiments, the probing molecule is an antibody.

In some embodiments, the target molecule comprises a peptide.

In some embodiments, the target molecule is selected from the group consisting of: a cytokine, a chemokine, an integrin, an adhesion molecule, and an immune checkpoint molecule.

In some embodiments, the agent is a drug affecting the target molecule.

In some embodiments, the agent comprises an antibody.

In some embodiments, coupled is in contact or at least partially overlapping.

In some embodiments, the device further comprises a detection unit in operable communication with the device, and wherein the detection unit is configured to detect the signal.

In some embodiments, the detection unit comprises an element selected form the group consisting of: an active-pixel sensor (APS), an electrode, an excitation source with active-pixel sensor, and any combination thereof

In some embodiments, the method further comprises a step of quantifying the amount of the analyte in a sample, comprising: determining the amount of the signal, and comparing it to a calibration curve or an indicative value, thereby quantifying the amount of the analyte in the sample.

In some embodiments, the analyte comprises an antibody.

In some embodiments, the antibody comprises an antibody drug, a neutralizing antibody of the antibody drug, or both.

In some embodiments, the drug comprises an immune checkpoint inhibitor.

In some embodiments, the drug targets a cytokine.

In some embodiments, the method further comprises a step of determining the amount of the drug in the sample.

In some embodiments, the sample is obtained or derived from a subject.

In some embodiments, determining the presence of the analyte comprises determining the presence, the amount, or both, of an antibody drug, a neutralizing antibody of the antibody drug, or both in the sample or a subject.

In some embodiments, the subject is afflicted with a cell proliferation related disease, an immune disease, or both.

In some embodiments, the cell proliferation related disease comprises cancer.

In some embodiments, the immune disease comprises an autoimmune disease, an inflammatory disease, or both.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a perspective view simplified illustration of a capture flow device, according to some embodiments of the present invention.

FIG. 2 includes a perspective view simplified illustration of how the capture flow device works during an assay measurement according to some embodiments of the present invention with an analyte absent from a sample (negative control).

FIG. 3 includes a perspective view simplified illustration of how the capture flow device works during an assay measurement according to some embodiments of the present invention with a sample comprising a non-affecting analyte.

FIG. 4 includes a perspective view simplified illustration of how the capture flow device works during an assay measurement according to some embodiments of the present invention with a sample comprising a neutralizing analyte.

FIG. 5 includes a perspective view simplified illustration of how the capture flow device works during an assay measurement according to some embodiments of the present invention with sample comprising both a neutralizing and a non-affecting analyte.

FIG. 6 includes a graph showing a calibration curve for neutralizing antibodies, using the capture flow device of the invention.

FIGS. 7A-7B include perspective view simplified illustrations of how the capture flow device works during an assay measurement according to some embodiments of the present invention with a sample comprising a neutralizing analyte and an analyte (7A) or a sample comprising an analyte and being devoid of a neutralizing analyte (7B).

FIGS. 8A-8B include perspective view simplified illustrations of how the capture flow device works during an assay measurement according to some embodiments of the present invention with a sample comprising a neutralizing analyte and an analyte (8A) or a sample comprising an analyte and being devoid of a neutralizing analyte (8B).

FIGS. 9A-9B include perspective view simplified illustrations of how the capture flow device works during an assay measurement according to some embodiments of the present invention with a sample comprising a neutralizing analyte and an analyte (9A) or a sample comprising an analyte and being devoid of a neutralizing analyte (9B).

FIGS. 10A-10B include perspective view simplified illustrations of how the capture flow device works during an assay measurement according to some embodiments of the present invention with a sample comprising a neutralizing analyte and an analyte (10A) or a sample comprising an analyte and being devoid of a neutralizing analyte (10B).

FIGS. 11A-11B include a photograph and a graph showing dose response of neutralizing anti-drug antibodies (nADA) on the substrate line when running the test. (11A) A photo of the results taken after the sample finished running in the assay. The picture was taken with a mobile phone. (11B) Graphical representation of the mean±STDEV of the substrate line's relative color intensity. The intensity of the line was calculated using Fiji software from (11A). Lateral flow direction is from bottom of image towards the top.

FIGS. 12A-12B include a photograph and a graph showing analysis of the capture lines after the end of the test. (12A) Picture of the capture lines taken with mobile phone. (12B) Graphical representation of the mean ±STDEV of the target molecule (e.g., Tumor necrosis factor alpha (TNFα)) capture line's (lowest line) relative color intensity. The color intensity of the line was calculated using Fiji software from the picture presented in (12A). Lateral flow direction is from bottom of image towards the top.

FIGS. 13A-13B include a photograph and a graph showing dose response of nADA on the substrate line when running the test. (13A) A photo of the results taken after the sample finished running in the assay. The picture was taken with a mobile phone. (13B) Graphical representation of the substrate line's relative color intensity. The intensity of the line was calculated using Fiji software from (13A). Lateral flow direction is from bottom of image towards the top.

FIGS. 14A-14B include a photograph and a graph showing dose response of drug on the drug capture line (“upper line”). (14A) A photo of the results taken after the sample finished running in the assay and DAB substrate was added on top of the membrane. The picture was taken with a mobile phone. (14B) Graphical representation of the mean ±STDEV of the drug capture line's relative color intensity. The intensity of the line was calculated using Fiji software from (14A). Lateral flow direction is from bottom of image towards the top.

FIGS. 15A-15B include a photograph and a graph showing dose response of nADA on the target molecule (e.g., TNFα) capture line (lower line). (15A) A photo of the results taken after the sample finished running in the assay and DAB substrate was added on top of the membrane. The picture was taken with a mobile phone. (15B) Graphical representation of the mean ±STDEV of the (e.g., TNFα) capture line's relative color intensity. The intensity of the line was calculated using Fiji software from (15A). Lateral flow direction is from bottom of image towards the top.

FIGS. 16A-16B include a photograph and a graph showing dose response of nADA on the TNF capture line (lower line). (16A) A photo of the results taken after the sample finished running in the assay and DAB substrate was added on top of the membrane. The picture was taken with a mobile phone. (16B) Graphical representation of the mean ±STDEV of the TNF capture line's relative color intensity. The intensity of the line was calculated using Fiji software from (16A). Lateral flow direction is from bottom of image towards the top.

### DETAILED DESCRIPTION

The present invention, in some embodiments, relates to a lateral flow device. In some embodiments, the device is a point of care testing device.

According to some embodiments, there is provided a device, comprising a section 1, at least one section 2, at least one section 3, and a section 4, wherein section 1 is coupled to at least one section 2, at least one section 3 is coupled to at least one section 2 and section 4 and comprises a surface functionalized with a target molecule and with an agent having specific binding affinity to the target molecule.

In some embodiments, section 4 is in contact with a substrate molecule.

In some embodiments, the device further comprises a section 5. In some embodiments, section 5 is in contact with a substrate molecule. In some embodiments, when the device comprises section 5, section 4 is devoid of a substrate molecule. In some embodiments, section 5 is coupled section 4.

In some embodiments, section 1, at least one section 2, at least one section 3, and section 4, are arranged along a horizontal axis and in liquid communication allowing lateral flow sequentially from the section 1 throughout all sections to the section 4. In some embodiments, section 1, at least one section 2, at least one section 3, and section 4 are arranged along a horizontal axis, wherein any subsequent section is in liquid communication or is coupled so as to allow a lateral flow sequentially from the section 1 throughout all sections to section 4.

According to some embodiments, there is provided a device, comprising at least a section 1, a section 2, a section 3, a section 4, and a section 5, wherein section 1 is coupled to section 2, section 3 is coupled to section 2 and section 4 and comprises a surface functionalized with a target molecule, section 4 is coupled to section 5 and is functionalized with an agent having specific binding affinity to the target molecule, and section 5 is in contact with a substrate molecule.

In some embodiments, section 1, section 2, section 3, section 4, and section 5 are arranged along a horizontal axis and in liquid communication allowing lateral flow sequentially from the section 1 throughout all sections to the section 5. In some embodiments, section 1, section 2, section 3, section 4, and section 5 are arranged along a horizontal axis, wherein any subsequent section is in liquid communication or is coupled so as to allow a lateral flow sequentially from the section 1 throughout all sections to section 5.

In some embodiments, section 1 comprises a sample collecting surface. In some embodiments, section 1 comprises a sample depositing surface.

In some embodiments, at least one section 2 comprises a surface comprising an agent and an agent probing molecule having specific binding affinity to the agent.

In some embodiments, at least one section 2 comprises 2 or more sections 2. In some embodiments, at least one section 2 comprises 2 sections 2. In some embodiments, the device comprises 2 sections 2. In some embodiments, a first section 2 comprises an agent as disclosed herein. In some embodiments, a second section 2 comprises agent probing molecule having specific binding affinity to the agent. In some embodiments, the first section 2 is coupled to section 1 and to the second section 2. In some embodiments, the second section 2 is coupled to section 1 and the first section 2. In some embodiments, the lateral flow is sequentially from the section 1 to the first section 2, the second section 2, and throughout all other sections as disclosed herein. In some embodiments, the lateral flow is sequentially from the section 1 to the second section 2, the first section 2, and throughout all other sections as disclosed herein.

In some embodiments, at least one section 3 comprises 2 or more sections 3. In some embodiments, at least one section 3 comprises 2 sections 3. In some embodiments, the device comprises 2 sections 3. In some embodiments, a first section 3 comprises a surface functionalized with a target molecule as disclosed herein. In some embodiments, a second section 3 comprises a surface functionalized with an agent having specific binding affinity to the target molecule. In some embodiments, the first section 3 is coupled to at least one section 2, as disclosed herein, and to section 4. In some embodiments, the second section 3 is coupled to at least one section 2, as disclosed herein, and section 4. In some embodiments, the lateral flow is sequentially from the at least one section 2, as disclosed herein, to the first section 3, the second section 3, and throughout all other sections as disclosed herein (e.g., section 4, or section 4 through to section 5). In some embodiments, the lateral flow is sequentially from the at least one section 2, as disclosed herein, to the second section 3, the first section 3, and throughout all other sections as disclosed herein (e.g., section 4, or section 4 through to section 5).

In some embodiments, the agent probing molecule is linked to a reporter molecule. In some embodiments, the reporter molecule generates a trigger. In some embodiments, the trigger generates a chemically and/or physically detectable reaction or signal.

In some embodiments, section 4 or 5 comprises a surface in contact with a substrate molecule, wherein the substrate molecule generates a detectable signal or reaction in response to the trigger generated by the reporter molecule linked to the probing molecule. In some embodiments, the substrate molecule is in the presence of an amplifier of a signal. In some embodiments, an amplifier of a signal increases the amount of signal generated and/or detected. In one embodiment, an amplifier of a signal comprises a gold particle.

As used herein, the term “coupled” comprises in contact with or in liquid communication.

The phrases “in liquid communication”, “in contact with”, and “coupled” are used herein interchangeably.

In some embodiments, section 1, at least one section 2, at least one section 3, and section 4 are partially overlapping. In some embodiments, section 1, at least one section 2, at least one section 3, and section 4 are partially overlapping, wherein overlapping comprises from 0.01% to 99%, from 0.01% to 95%, from 0.01% to 90%, from 1% to 90%, from 0.01% to 1%, from 1% to 80%, from 1% to 70%, from 1% to 60%, from 1% to 50%, from 1% to 40%, from 1% to 30%, from 1% to 20%, from 1% to 10%, from 1% to 5%, from 5% to 10%, from 10% to 20%, from 20% to 30%, from 30% to 40%, from 10% to 30%, from 10% to 40%, from 10% to 50%, from 10% to 60%, from 10% to 70%, of the total surface of the section.

In some embodiments, a first section 2 and a second section 2 are partially overlapping. In some embodiments, a first section 2 and a second section 2 are partially overlapping, wherein overlapping comprises from 0.01% to 99%, from 0.01% to 95%, from 0.01% to 90%, from 1% to 90%, from 0.01% to 1%, from 1% to 80%, from 1% to 70%, from 1% to 60%, from 1% to 50%, from 1% to 40%, from 1% to 30%, from 1% to 20%, from 1% to 10%, from 1% to 5%, from 5% to 10%, from 10% to 20%, from 20% to 30%, from 30% to 40%, from 10% to 30%, from 10% to 40%, from 10% to 50%, from 10% to 60%, from 10% to 70%, of the total surface of the section.

In some embodiments, a first section 3 and a second section 3 are partially overlapping. In some embodiments, a first section 3 and a second section 3 are partially overlapping, wherein overlapping comprises from 0.01% to 99%, from 0.01% to 95%, from 0.01% to 90%, from 1% to 90%, from 0.01% to 1%, from 1% to 80%, from 1% to 70%, from 1% to 60%, from 1% to 50%, from 1% to 40%, from 1% to 30%, from 1% to 20%, from 1% to 10%, from 1% to 5%, from 5% to 10%, from 10% to 20%, from 20% to 30%, from 30% to 40%, from 10% to 30%, from 10% to 40%, from 10% to 50%, from 10% to 60%, from 10% to 70%, of the total surface of the section.

In some embodiments, section 1, section 2, section 3, section 4, and section 5 are partially overlapping. In some embodiments, section 1, section 2, section 3, section 4, and section 5 are partially overlapping, wherein overlapping comprises from 0.01% to 99%, from 0.01% to 95%, from 0.01% to 90%, from 1% to 90%, from 0.01% to 1%, from 1% to 80%, from 1% to 70%, from 1% to 60%, from 1% to 50%, from 1% to 40%, from 1% to 30%, from 1% to 20%, from 1% to 10%, from 1% to 5%, from 5% to 10%, from 10% to 20%, from 20% to 30%, from 30% to 40%, from 10% to 30%, from 10% to 40%, from 10% to 50%, from 10% to 60%, from 10% to 70%, of the total surface of the section.

In some embodiments of the present invention there is provided a device, comprising: (i) a section 1 comprising a sample collecting surface, (ii) at least one section 2 comprising a surface comprising an agent and an agent probing molecule linked to a reporter molecule, wherein the agent probing molecule has specific affinity to the agent, and wherein the signal molecule is configured to generate a chemically and/or a physically detectable signal, (iii) at least one section 3 comprising a surface functionalized with a target molecule and with an agent having specific binding affinity to the target molecule, and (iv) a section 4 comprising a surface functionalized with, and (v) a section 5 comprising a surface with a substrate molecule deposited thereon, wherein the substrate molecule produces or is converted to a detectable signal when in contact with the reporter molecule, and wherein sections 1, 2, 3, 4 and 5 are arranged along a horizontal axis and in liquid communication allowing lateral flow sequentially from the section 1 throughout all sections to the section 5.

In some embodiments of the present invention there is provided a device, comprising: (i) a section 1 comprising a sample collecting surface, (ii) a section 2 comprising a surface comprising an agent probing molecule linked to a reporter molecule, wherein the agent probing molecule has specific affinity to an agent, and wherein the signal molecule is configured to generate a chemically and/or a physically detectable signal, (iii) a section 3 comprising a surface functionalized with a target molecule, (iv) a section 4 comprising a surface functionalized with an agent having specific binding affinity to the target molecule, and (v) a section 5 comprising a surface with a substrate molecule deposited thereon, wherein the substrate molecule produces or is converted to a detectable signal when in contact with the reporter molecule, and wherein sections 1, 2, 3, 4 and 5 are arranged along a horizontal axis and in liquid communication allowing lateral flow sequentially from the section 1 throughout all sections to the section 5.

In some embodiments, there is provided a device for determining the presence of an analyte in a sample. In some embodiments, there is provided a device for quantifying the amount of an analyte in a sample. In some embodiments, there is provided a device for determining and quantifying the amount of an analyte in a sample. In some embodiments, quantitation is relative or absolute.

In some embodiments, there is provided a lateral flow device. In some embodiments, a device according to the present invention is a point of care testing device.

As used herein the phrase “lateral flow device” refers to any device including a bibulous or non-bibulous matrix, which is capable of transporting an analyte and/or a reagent to a pre-selected site. Many such devices are known, in which the strip is made of water absorbing materials such as nitrocellulose, paper, cellulose, and other bibulous materials. A test strip used in lateral flow, is a strip in which a test sample suspected of containing an analyte flows through the strip to a detection zone in which the analyte (if present) interacts with a detection agent to indicate a presence, absence and/or quantity of the analyte.

In some embodiments, a lateral flow device comprises a microfluidic device.

As used herein, the terms “microfluidic device” or “microfluidics” encompasses any device which applies fluid flow to paths, e.g., channels, being smaller than 1 mm in at least one of their dimensions.

As used herein, the term “point of care testing” refers to real time diagnostic testing that can be done in a rapid time frame so that the resulting test is performed faster than comparable tests that do not employ this system. It can be performed in a doctor's office, at a bedside, in a laboratory, a clinic, an emergency room, ambulances, at home or other such locales, particularly where rapid and accurate results are required. The patient can be present, but such presence is not required. Point of care includes, but is not limited to, emergency rooms, operating rooms, hospital laboratories and other clinical laboratories, doctor's offices, in the field, or in any situation in which a rapid and accurate result is desired.

As used herein the term “analyte” refers to a substance to be detected which may be present in a test sample. The analyte can be any substance for which there exists a naturally occurring specific binding member (such as, an antibody or aptamer, DNA, etc..), or for which a specific binding member can be prepared. Thus, an analyte is a substance that can bind to one or more specific binding members in an assay. “Analyte” also includes any antigenic substance, hapten, antibody, and combinations thereof. As a member of a specific binding pair the analyte can be detected by means of naturally occurring specific binding partners (pairs). It is to be understood that the invention can be configured for detecting a broad range of analytes, including inhibitors of therapeutic drugs.

In some embodiments, the analyte comprises or consists of a therapeutic drug. In some embodiments, the analyte binds to a target molecule.

In some embodiments, the analyte comprises or consists of a therapeutic drug inhibitor. In some embodiments, the analyte binds to a drug. In some embodiments, the analyte inhibits, reduces, hampers, or any combination thereof, the activity, efficacy, or both, of the drug. In some embodiments, the analyte is an anti-drug antibody. In some embodiments, the anti-drug antibody is an anti-drug neutralizing antibody.

As used herein, the term “neutralizing” means the anti-drug antibody renders the drug inactive or partially inactive.

As used herein, “drug activity” encompasses any action that a drug exerts on a target molecule, including, but not limited to, binding to the target molecule, degrading the target molecule, chemically modifying the target molecule, competing with the target molecule, e.g., with a binding counterpart, such as a receptor, or others. In some embodiments, “drug activity” encompasses any action exerts by a drug on its target which reduces or inhibits a signaling pathway comprising the target molecule.

In some embodiments, an anti-drug neutralizing antibody is produced by a subject administered with a drug. In some embodiments, the administered drug induces an immunogenic response in a subject. In some embodiments, the administered drug induces or elicits the production of antibodies targeting the drug. In some embodiments, the anti- drug antibody is a neutralizing antibody or a non-neutralizing antibody. In some embodiments, a subject administered with the drug produces an anti-drug neutralizing antibody, an anti-drug non-neutralizing antibody, or a combination thereof. In some embodiments, the serum of a subject administered with the drug comprises an anti-drug neutralizing antibody, a drug non-neutralizing antibody, or a combination thereof.

In some embodiments, the subject is an animal subject. In some embodiments, the subject is a mammal. In some embodiments, the subject is a human subject.

In some embodiments, the subject is afflicted with a cell proliferation related disease, an immune disease, or both.

In some embodiments, a cell proliferation related disease comprises cancer.

In some embodiments, an immune disease comprises an autoimmune disease, an inflammatory disease, or both.

In some embodiments, the anti-drug neutralizing antibody has increased binding affinity to the drug. In some embodiments, the anti-drug neutralizing antibody is capable of reducing the efficacy of the drug. In some embodiments, the anti-drug neutralizing antibody is capable of increasing the clearance of the drug, targeting the binding site of the drug, or both. In some embodiments, an anti-drug neutralizing antibody comprises any antibody capable of preventing, blocking, inhibiting, or any combination thereof, the interaction between a drug and its target molecule.

As used herein, the phrase “binding site” refers to any residue, moiety, portion, surface, part, or any combination or equivalent thereof, within the drug which is in contact with a target molecule (e.g., when the neutralizing antibody is absent).

In some embodiments, the sample is a biological sample. In some embodiments, the sample is suspicious of comprising an analyte as describe herein. In some embodiments, the sample is obtained or derived from a subject. In some embodiments, the sample comprises bodily fluids. In some embodiments, the sample comprises a blood sample (e.g., whole blood). In some embodiments, the sample comprises serum or any equivalent thereof (e.g., any fraction of blood or an equivalent thereof, wherein antibodies or molecules equivalent thereto are present). In some embodiments, the sample is an ex-vivo sample.

Methods for obtaining biological samples, e.g., blood samples and/or serum therefrom, are common and would be apparent to one of ordinary skill in the art.

In some embodiments, of the present invention, there is provided a device which utilizes counterparts with specific binding to each other, e.g., increased affinity.

As used herein, the phrase “specific binding counterparts” refers to any member of a specific binding complex. In some embodiments, the binding complex comprises at least two, at least 3, at least 4, at least 5, at least 7, or at least 10 counterparts, or any value and range there between. Each possibility represents a separate embodiment of the invention. In some embodiments, the binding complex comprises 2 to 5, 3 to 8, 2 to 10, or 3 to 7 counterparts. Each possibility represents a separate embodiment of the invention.

In some embodiments, at least one of the specific binding counterparts binds to at least another counterpart of the binding complex through chemical or physical means.

In some embodiments, the binding complex comprises at least one antigen and at least one antibody. In some embodiments the antigen is an antibody.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelid, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulfide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “substrate molecule” as used herein, refers to a molecule that interact specifically with a reporter molecule. By “interacts specifically” it is meant that the substrate molecule exhibits essentially a structural or physical change leading to the generation of a detectable and/or measurable physical signal.

The term “specificity” as used herein, refers to the ability of a binding moiety to bind preferentially or predominantly to one counterpart molecule, versus a different counterpart molecule, and does not necessarily imply high affinity (as defined further herein).

The term “affinity”, as used herein, refers to the degree to which a first compound, e.g., an anti-drug neutralizing antibody, binds to a second molecule, e.g., a drug, so as to shift the equilibrium of the free second compound toward the presence of a complex formed by their binding. Thus, for example, where an analyte comprising an anti-drug neutralizing antibody and a drug molecule are combined in relatively equal concentration, an anti-drug neutralizing antibody of high affinity will bind to the available drug molecule so as to shift the equilibrium toward high concentration of the resulting complex. The dissociation constant (Kd) is commonly used to describe the affinity between, e.g., the anti-drug neutralizing antibody, and its targeted or affected drug. In some embodiments, the dissociation constant is lower than 10⁻² M. In some embodiments, the dissociation constant is lower than 10⁻³ M. In some embodiments, the dissociation constant is lower than 10⁻⁴ M. In some embodiments, the dissociation constant is lower than 10⁻⁵ M. In some embodiments, the dissociation constant is lower than 10⁻⁶ M. In some embodiments, the dissociation constant is lower than 10⁻⁷ M. In some embodiments, the dissociation constant is lower than 10⁻⁸ M. In some embodiments, the dissociation constant is lower than 10⁻⁹ M.

The terms “specifically bind” and “specific binding”, as used herein, refer to the ability of a binding domain to preferentially or predominantly bind to a particular molecule that is present in a mixture of different molecules. In some embodiments, a specific binding interaction will discriminate between desirable and undesirable molecules in a sample.

As used herein, the term “functionalized surface” refers to a surface of an article that has been modified so that one or a plurality of molecules or functional groups are present thereon. In some embodiments, the plurality of molecules or functional groups are bound to the functionalized surface. The manner by which functionalization is achieved depends on, for example, the nature of the chemical compound and the nature and composition of the surface.

As used herein, the term “surface” refers to the material that the sections of the invention are made of. In some embodiments, surface refers to an outer surface. A variety of materials can be used as surface according to the present invention. The materials include any material that can act as a support for attachment of the molecules of interest. Such materials are known to those of skill in this art. These materials include, but are not limited to, organic or inorganic polymers, natural and synthetic polymers, including, but not limited to, agarose, cellulose, nitrocellulose, cellulose acetate, other cellulose derivatives, dextran, dextran-derivatives and dextran co-polymers, other polysaccharides, glass, silica gels, gelatin, polyvinyl pyrrolidone, rayon, nylon, polyethylene, polypropylene, polybutylene, polycarbonate, polyesters, polyamides, vinyl polymers, polyvinyl alcohol (PVA), polystyrene and polystyrene copolymers, polystyrene cross-linked with divinylbenzene or the like, acrylic resins, acrylates and acrylic acids, acrylamides, polyacrylamides, polyacrylamide blends, co-polymers of vinyl and acrylamide, methacrylate, methacrylate derivatives and co-polymers, other polymers and co-polymers with various functional groups, latex, butyl rubber and other synthetic rubbers, silicon, glass, paper, natural sponges, insoluble protein, surfactants, red blood cells, metals, metalloids, magnetic materials, or other commercially available media. In some embodiments, the surface comprises a water absorbing material, as described hereinabove.

### Device and Assay

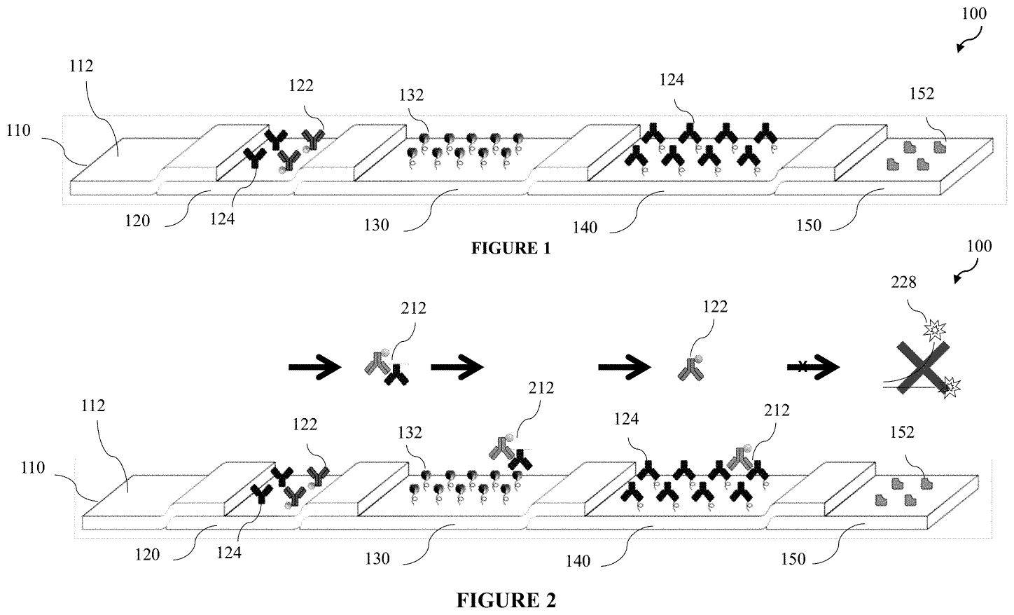

Reference is made to FIG. 1, which is a simplified illustration of some of the components of a device 100, according to some embodiments of the invention.

According to some embodiments of the present invention, section 1 110, section 2 120, section 3 130, section 4 140, and section 5 150 are arranged along a horizontal axis and in liquid communication allowing lateral flow from section 1 throughout all sections to section 5.

In some embodiments, section 1 110, section 2 120, section 3 130, and section 4 140, are in contact with each other, so as to allow a lateral flow from section 1 throughout all sections to section 4.

In some embodiments, section 1 110, section 2 120, section 3 130, section 4 140, and section 5 150 are partially overlapping. In some embodiments, overlapping is in the range of 0.01% to 99% of the total surface of a section. In some embodiments, overlapping is in the range of 0.05% to 99%, 0.1% to 99%, 0.1% to 90%, 0.1% to 80%, 0.1% to 70%, 0.1% to 60%, 0.1% to 50%, 0.1% to 40%, 0.1% to 30%, 0.1% to 29%, 0.1% to 10%, or 0.1% to 5% of the total surface of a section, including any range therebetween. In some embodiments, section 1 is partially overlapping above section 2. In some embodiments, section 1 is partially overlapping below section 2. In some embodiments, section 2 is partially overlapping below section 3. In some embodiments, section 2 is partially overlapping above section 3. In some embodiments, section 3 is partially overlapping above section 4. In some embodiments, section 3 is partially overlapping below section 4. In some embodiments, section 4 is partially overlapping above section 5. In some embodiments, section 4 is partially overlapping below section 5.

In some embodiments, at least four sections of a device according to the present invention are disposed along more than one plane. In some embodiments, two consecutive sections are disposed along one or more planes. In some embodiments, section 1 110, section 2 120, section 3 130, section 4 140, and section 5 150 share at least one plane. In some embodiments, all sections are disposed along the same plane.

According to some embodiments of the present invention, section 1 110, section 2 120, section 3 130, section 4 140, and section 5 150 serve as solid support onto which different components are either adsorbed or immobilized (such as bound). In some embodiments, section 2 120, section 3 130, section 4 140, and section 5 150 comprise a surface in contact with or bound to a component (such as an analyte, an agent, an agent probing molecule, a target molecule, and a substrate molecule), wherein the surface is as described hereinabove. In some embodiments, section 2 120, comprises an agent 124 and an agent probing molecule 122 (such as an antibody) adsorbed or deposited thereon. In some embodiments, the component on section 3 130 comprise a target molecule 132 covalently immobilized (e.g. covalently bound) to the section. In some embodiments, the different components are deposited or adsorbed prior to the assembly of the sections. In some embodiments, the different components are deposited or adsorbed after the assembly of the sections. In some embodiments, the different components are immobilized prior to the assembly of the sections. In some embodiments, the different components are immobilized after the assembly of the sections.

In some embodiments, section 1 110 comprises a sample collecting surface 112, section 2 120 comprises a surface comprising an agent 124 and an agent probing molecule 122, section 3 130 comprises a surface functionalized with a target molecule 132, section 4 140 comprises a surface functionalized with an agent 124, and section 5 150 comprises a substrate molecule deposited thereon 152.

In some embodiments, section 1 110, section 2 120, section 3 130, section 4 140, and section 5 150 comprise a membrane. In some embodiments, a membrane comprises polyester. In some embodiments, a membrane comprises cellulose. In one embodiment, cellulose membrane comprises nitrocellulose membrane.

As used herein the term “membrane” refers to a boundary, a layer, barrier, or material, which may, or may not be permeable. The term “membrane” may further refer to an interface. In some embodiments, the terms “membrane” and “surface” are used herein interchangeably. Unless specified otherwise, membranes may take the form a solid, liquid, or gel, and may or may not have a distinct lattice, none cross-linked structure, or cross-linked structure. In some embodiments, the membrane is a fibrous membrane.

In some embodiments, section 1 110, section 2 120, section 3 130, section 4 140, and section 5 150 comprise a matrix. The matrix defines a lateral flow path. In some embodiments, the path is a microfluidic path. In some embodiments, the flow path is axial, and the flow is unidirectional. In some embodiments, the flow direction is downstream from section 1. As used herein the term “downstream” refers to a location or direction to which liquid that is applied or deposited on the sample collecting surface will flow, such location or direction being on the opposite direction to section 1. In some embodiments, the dissolved or dispersed components of the liquid sample are carried at substantially equal rates and with relatively unimpaired flow laterally through the matrix. In some embodiments, the lateral flow as used herein, refers to a capillary flow. In some embodiments, the lateral flow is generated by a capillary action. In some embodiments, the dissolved or dispersed components of the liquid sample are modulated by the added PVA membrane and other surface-active materials or ionic buffers forces.

Typical matrix materials that can be used in a device according to the present invention include high density polyethylene, nitrocellulose, polyvinyl chloride, polyvinyl acetate, copolymers of vinyl acetate and vinyl chloride, polyamide, polycarbonate, nylon, glass fiber, orlon, polyester, polystyrene, cotton, cellulose and the like, or blends. The optimum pore diameter for the membrane for use in the invention is about 20 μm to about 140 μm. Other materials, such as untreated paper, derivatized nylon, cellulose and the like may also be used according to the present invention.

In some embodiments, the matrix or the membrane comprises a hydrophilic material. In some embodiments, the hydrophilic material is a hydrophilic polymer. In some embodiments, the matrix or the membrane comprises a polymer wettable by an aqueous solution.

Reference is now made to FIG. 2, which is a simplified illustration of how a device 100 works during an assay measurement according to some embodiments of the present invention, in the case of a negative control (no sample is loaded). In some embodiments, the device comprises a section 1 110 with a sample collecting surface 112, a section 2 120 comprising a surface with an agent 124 and an agent probing molecule 122 linked to a reporter molecule, a section 3 130 comprising surface functionalized with a target molecule 132, a section 4 140 functionalized with an agent 124, and section 5 150 comprising a surface with a substrate molecule 152 deposited or adsorbed thereto.

In some embodiments, a liquid sample devoid of an analyte is placed in section 1 110. The sample migrates to section 2 120, via lateral flow, where it encounters the agent 124 and the agent probing molecule 122. A complex 212 is formed based on molecular recognition (such as affinity-based interaction or binding between an antigen, e.g., the agent, and an antibody, e.g., the agent probing molecule), wherein complex 212 comprises the agent 124 bound or in contact with the agent probing molecule 122, and wherein the agent probing molecule 122 is bound to a reporter molecule generating a trigger. The complex formed 212, continues to migrate via lateral flow to section 3 130 comprising the target molecule 132. The complex formed 212 will be immobilized in section 3 130 and will not continue and migrate to section 4 140. Any excess of agent probing molecule 122 which is not a part of a complex 212 will migrate unbound with the sample to section 4 140 comprising a surface functionalized with the agent 124, where it will link to agent 124, forming the complex 212 and stopped from migrating further to the next section, being section 5 150, thus no visible signal will be observed in section 5 150 (as exemplified in FIG. 2 by the “cross” on signal 228).

In some embodiments, section 1 110, section 2 120, section 3 130, section 4 140, and section 5 150 are arranged in such way that section 3 130 is able to receive both agent-agent probing molecule (comprising the reporter molecule) complex 212 and excess of free agent 124, and section 4 140 is able to receive only free agent probing molecule 122.

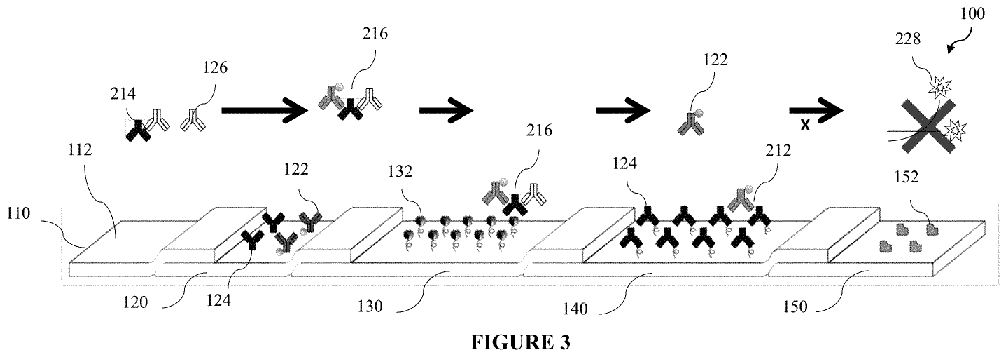

Reference is now made to FIG. 3, which is a simplified illustration of how a device 100 works during an assay measurement according to some embodiments of the present invention, in the case of a sample comprising an analyte. In some embodiments, the device comprises a section 1 110 with a sample collecting surface 112, a section 2 120 comprising a surface with an agent 124 and an agent probing molecule 122 linked to a reporter molecule, a section 3 130 comprising surface functionalized with a target molecule 132, a section 4 140 functionalized with an agent 124, and section 5 150 comprising a surface with a substrate molecule 152 deposited or adsorbed thereto.

In some embodiments, a liquid sample comprising an analyte comprising a non-neutralizing anti-agent antibody 126, and being devoid of a neutralizing anti-agent antibody, and/or a complex 214 comprising the agent 124 bound or in contact with the non-neutralizing anti-agent antibody 126, is deposited in section 1 110. The sample migrates to section 2 120, via lateral flow, where it encounters the agent 124 and the agent probing molecule 122. A complex 216 is formed based on molecular recognition (such as affinity-based interaction or binding, as described herein), wherein complex 216 comprises the agent 124 bound or in contact with both the agent probing molecule 122 and with the non-neutralizing anti-agent antibody 126, and wherein the agent probing molecule 122 is bound to a reporter molecule generating a trigger. The complex formed 216, continues to migrate via lateral flow to section 3 130 comprising the target molecule 132. The complex formed 216 will be immobilized in section 3 130 and will not continue and migrate to section 4 140. Any excess of agent probing molecule 122 which is not a part of a complex 216 will migrate unbound with the sample to section 4 140 comprising a surface functionalized with the agent 124, where it will link to agent 124, forming the complex 212 and stopped from migrating further to the next section, being section 5 150, thus no visible signal will be observed in section 5 150 (as exemplified in FIG. 3 by the “cross” on signal 228).

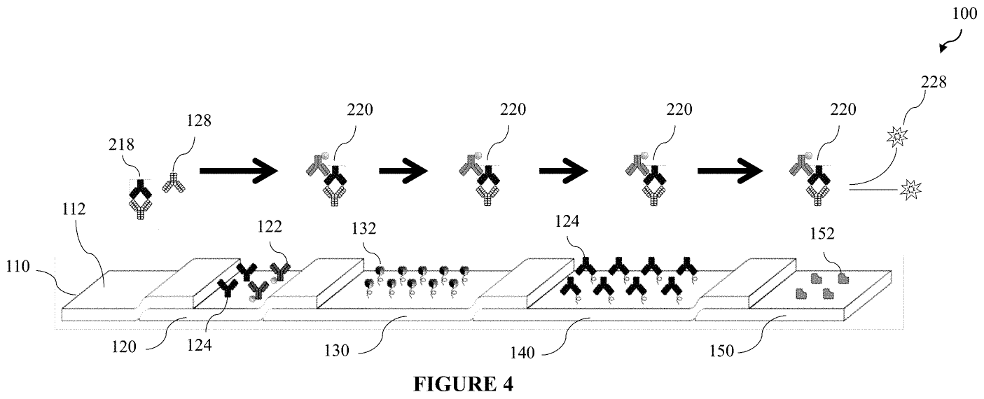

Reference is now made to FIG. 4, which is a simplified illustration of how a device 100 works during an assay measurement according to some embodiments of the present invention, in the case of a sample comprising an analyte. In some embodiments, the device comprises a section 1 110 with a sample collecting surface 112, a section 2 120 comprising a surface with an agent 124 and an agent probing molecule 122 linked to a reporter molecule, a section 3 130 comprising surface functionalized with a target molecule 132, a section 4 140 functionalized with an agent 124, and section 5 150 comprising a surface with a substrate molecule 152 deposited or adsorbed thereto.

In some embodiments, a liquid sample comprising an analyte comprising a neutralizing anti-agent antibody 128, and/or a complex 218 comprising the agent 124 bound or in contact with the neutralizing anti-agent antibody 128, is deposited in section 1 110. The sample migrates to section 2 120, via lateral flow, where it encounters the agent 124 and the agent probing molecule 122. A complex 220 is formed based on molecular recognition (such as affinity-based interaction or binding, as described herein), wherein complex 220 comprises the agent 124 bound or in contact with both the agent probing molecule 122 and with the neutralizing anti-agent antibody 128, and wherein the agent probing molecule 122 is bound to a reporter molecule generating a trigger. The complex formed 220, continues to migrate via lateral flow to section 3 130 comprising the target molecule 132. The complex formed 220 will not be immobilized in section 3 130 since the binding site of the agent is occupied by the neutralizing anti-agent antibody and it will continue and migrate to section 5 150 comprising a surface with the substrate molecule 152 adsorbed or deposited thereon. Here, the complex 220 or the trigger generated by the reporter molecule will interact with the substrate molecule 152, thereby generating a signal 228, and confirming the presence of the analyte in the sample. The type of signal generation will depend on the reporter molecule used that is conjugated to the reporter molecule and the substrate molecule deposited in section 5 150.

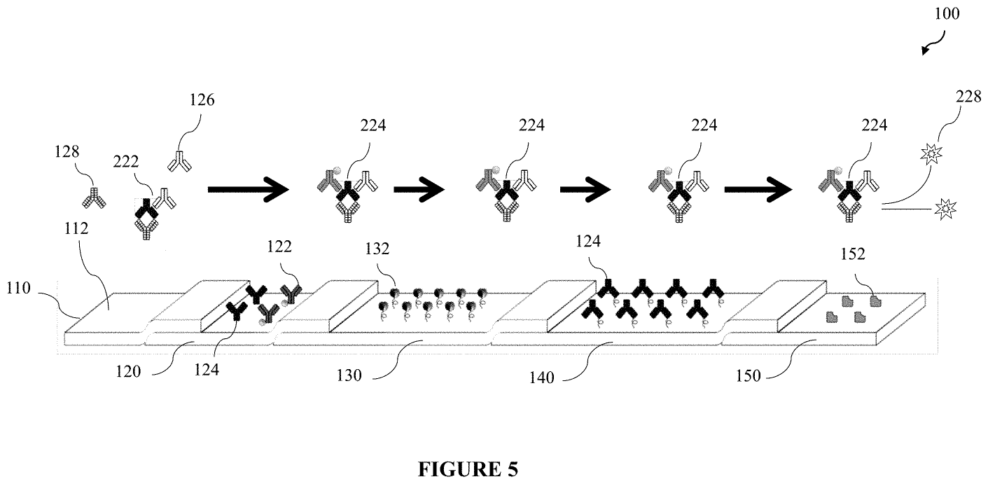

Reference is now made to FIG. 5, which is a simplified illustration of how a device 100 works during an assay measurement according to some embodiments of the present invention, in the case of a sample comprising an analyte. In some embodiments, the device comprises a section 1 110 with a sample collecting surface 112, a section 2 120 comprising a surface with an agent 124 and an agent probing molecule 122 linked to a reporter molecule, a section 3 130 comprising surface functionalized with a target molecule 132, a section 4 140 functionalized with an agent 124, and section 5 150 comprising a surface with a substrate molecule 152 deposited or adsorbed thereto.

In some embodiments, a liquid sample comprising an analyte comprising a neutralizing anti-agent antibody 128, a non-neutralizing anti-agent antibody 126, and/or a complex 222 comprising the agent 124 bound or in contact with both the neutralizing anti-agent antibody 128 the non-neutralizing anti-agent antibody 126, is deposited in section 1 110. The sample migrates to section 2 120, via lateral flow, where it encounters the agent 124 and the agent probing molecule 122. A complex 224 is formed based on molecular recognition (such as affinity-based interaction or binding, as described herein), wherein complex 224 comprises the agent 124 bound or in contact with the agent probing molecule 122, with the neutralizing anti-agent antibody 128, and the non-neutralizing anti-agent antibody 126, and wherein the agent probing molecule 122 is bound to a reporter molecule generating a trigger. The complex formed 224, continues to migrate via lateral flow to section 3 130 comprising the target molecule 132. The complex formed 224 will not be immobilized in section 3 130 since the binding site of the agent is occupied by the neutralizing anti-agent antibody and it will continue and migrate to section 5 150 comprising a surface with the substrate molecule 152 adsorbed or deposited thereon. Here, the complex 224 or the trigger generated by the reporter molecule will interact with the substrate molecule 152, thereby generating a signal 228, and confirming the presence of the analyte in the sample. The type of signal generation will depend on the reporter molecule used that is conjugated to the reporter molecule and the substrate molecule deposited in section 5 150.

In some embodiments, section 3 130 comprising a target molecule 132, positioned between section 2 120 and section 5 150, ensures that only complexes comprising the agent 124 bound to the neutralizing anti-agent antibody 128, e.g., complexes 220 and 224 as described hereinabove, will migrate to section 5 150.

In some embodiments, section 4 140 comprising an agent 124 positioned prior to or before section 5 150, ensures that excess of agent probing molecule 122 will not reach section 5 150. excess of agent probing molecule 122 will not reach section 5 150.

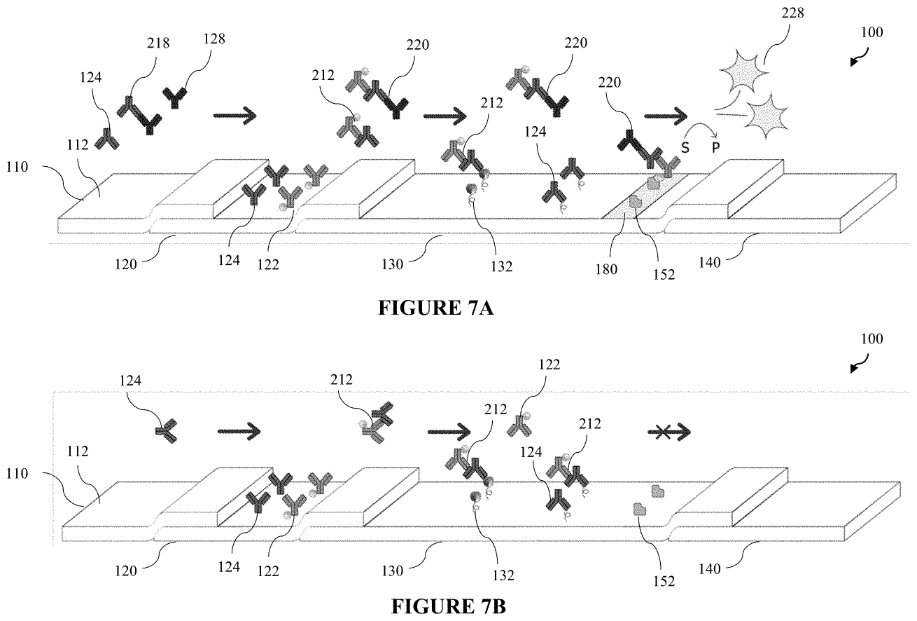

Reference is now made to FIG. 7, which is a simplified illustration of how a device 100 works during an assay measurement according to some embodiments of the present invention, in the case of a sample comprising an analyte. In some embodiments, the device comprises a section 1 110 with a sample collecting surface 112, a section 2 120 comprising a surface with an agent 124 and an agent probing molecule 122 linked to a reporter molecule, a section 3 130 comprising surface functionalized with a target molecule 132, an agent 124, and a substrate molecule 152 deposited or adsorbed thereto, and a section 4 140.

Reference is now made to FIG. 7A. In some embodiments, a liquid sample comprising an analyte comprising a neutralizing anti-agent antibody 128 , and/or a complex 218 comprising the agent 124 bound or in contact with the neutralizing anti-agent antibody 128, is deposited in section 1 110. The sample migrates to section 2 120, via lateral flow, where it encounters the agent 124 and the agent probing molecule 122. A complex 220 is formed based on molecular recognition (such as affinity-based interaction or binding, as described herein), wherein complex 220 comprises the agent 124 bound or in contact with the agent probing molecule 122, and with the neutralizing anti-agent antibody 128, and wherein the agent probing molecule 122 is bound to a reporter molecule generating a trigger. A complex 212 comprising the agent 124 bound or in contact with the agent probing molecule 122 may/is also formed. The complex formed 220, continues to migrate via lateral flow to section 3 130 comprising the target molecule 132. The complex formed 220 will not interact with the target molecule 132 and will continue to migrate through section 3 130. The complex formed 220 will interact with the substrate molecule 152 deposited on the “substrate line” 180, thereby generating a signal 228, and confirming the presence of the analyte, e.g., the neutralizing anti-drug antibody 128 in the sample. The type of signal generation will depend on the reporter molecule used that is conjugated to the reporter molecule and the substrate molecule deposited in section 3 130. The complex 212 comprising the agent 124 bound or in contact with the agent probing molecule 122 will bound to the target molecule 132 and therefore immobilized to section 3 130.

Reference is now made to FIG. 7B. In some embodiments, a liquid sample comprising an analyte devoid of a neutralizing anti-agent antibody 128 , and/or a complex 218 comprising the agent 124 bound or in contact with the neutralizing anti-agent antibody 128, is deposited in section 1 110. The sample migrates to section 2 120, via lateral flow, where it encounters the agent 124 and the agent probing molecule 122. A complex 212 comprising the agent 124 bound or in contact with the agent probing molecule 122 is formed. The complex 212 comprising the agent 124 bound or in contact with the agent probing molecule 122 will migrate to section 3 130 where it will bound to the target molecule 132 and, therefore immobilized thereto. Therefore, the complex formed 212 will not interact with the substrate molecule 152 deposited and a signal is not produced or formed.

In some embodiments, section 3 130 comprising a target molecule 132, positioned between section 2 120 and the substrate molecule 152 deposited on section 3 130, ensures that only complexes comprising the agent 124 bound to the neutralizing anti-agent antibody 128, e.g., complex 220 as described hereinabove, will migrate to the substrate molecule 152 deposited on the “substrate line” 180.

In some embodiments, section 3 130 comprising an agent 124 positioned prior to or before the substrate molecule 152 deposited on the “substrate line” 180, ensures that excess of agent probing molecule 122 will not reach the substrate molecule 152 deposited on the “substrate line” 180.

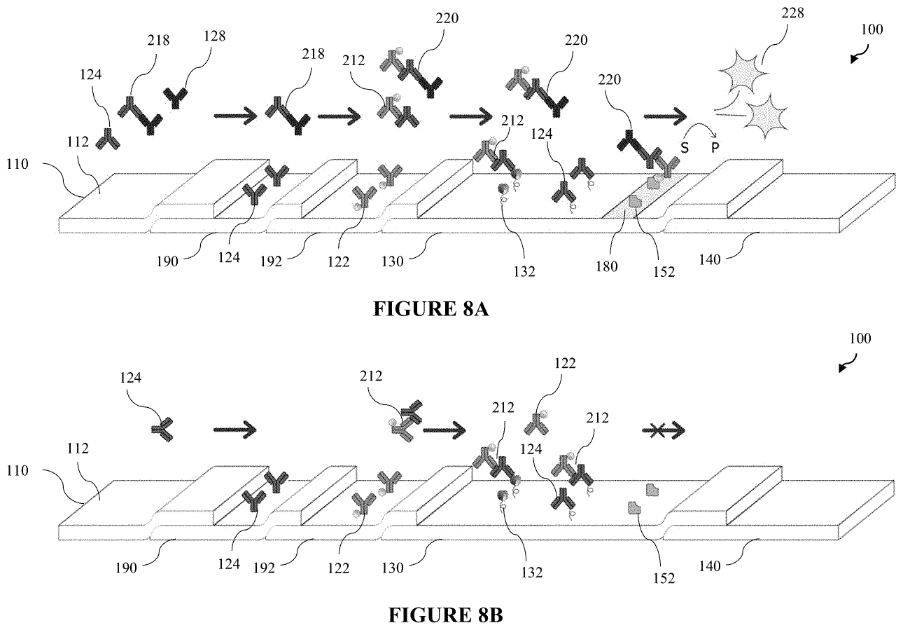

Reference is now made to FIG. 8, which is a simplified illustration of how a device 100 works during an assay measurement according to some embodiments of the present invention, in the case of a sample comprising an analyte. In some embodiments, the device comprises a section 1 110 with a sample collecting surface 112, a first section 2 190 comprising a surface with an agent 124, a second section 2 192 comprising a surface with an agent probing molecule 122 linked to a reporter molecule, a section 3 130 comprising surface functionalized with a target molecule 132, an agent 124, and a substrate molecule 152 deposited or adsorbed thereto, and a section 4 140.

Reference is now made to FIG. 8A. In some embodiments, a liquid sample comprising an analyte comprising a neutralizing anti-agent antibody 128 , and/or a complex 218 comprising the agent 124 bound or in contact with the neutralizing anti-agent antibody 128, is deposited in section 1 110. The sample migrates to the first section 2 190, via lateral flow, where it encounters the agent 124 and thereafter via lateral flow to the second section 2 192, where it encounters the agent probing molecule 122. A complex 220 is formed based on molecular recognition (such as affinity-based interaction or binding, as described herein), wherein complex 220 comprises the agent 124 bound or in contact with the agent probing molecule 122, and with the neutralizing anti-agent antibody 128, and wherein the agent probing molecule 122 is bound to a reporter molecule generating a trigger. A complex 212 comprising the agent 124 bound or in contact with the agent probing molecule 122 may/is also formed. The complex formed 220, continues to migrate via lateral flow to section 3 130 comprising the target molecule 132. The complex formed 220 will not interact with the target molecule 132 and will continue to migrate through section 3 130. The complex formed 220 will interact with the substrate molecule 152 deposited on the “substrate line” 180, thereby generating a signal 228, and confirming the presence of the analyte, e.g., the neutralizing anti-drug antibody 128 in the sample. The type of signal generation will depend on the reporter molecule used that is conjugated to the reporter molecule and the substrate molecule deposited in section 3 130. The complex 212 comprising the agent 124 bound or in contact with the agent probing molecule 122 will bound to the target molecule 132 and therefore immobilized to section 3 130.

Reference is now made to FIG. 8B. In some embodiments, a liquid sample comprising an analyte devoid of a neutralizing anti-agent antibody 128 , and/or a complex 218 comprising the agent 124 bound or in contact with the neutralizing anti-agent antibody 128, is deposited in section 1 110. The sample migrates to the first section 2 190, via lateral flow, where it encounters the agent 124 and thereafter via lateral flow to the second section 2 192, where it encounters the agent probing molecule 122. A complex 212 comprising the agent 124 bound or in contact with the agent probing molecule 122 is formed. The complex 212 comprising the agent 124 bound or in contact with the agent probing molecule 122 will migrate to section 3 130 where it will bound to the target molecule 132 and, therefore immobilized thereto. Therefore, the complex formed 212 will not interact with the substrate molecule 152 deposited and a signal is not produced or formed.

In some embodiments, section 3 130 comprising a target molecule 132, positioned between the second section 2 192 and the substrate molecule 152 deposited of section 3 130, ensures that only complexes comprising the agent 124 bound to the neutralizing anti-agent antibody 128, e.g., complex 220 as described hereinabove, will migrate to the substrate molecule 152 deposited on the “substrate line” 180.

In some embodiments, section 3 130 comprising an agent 124 positioned prior to or before the substrate molecule 152 deposited on the “substrate line” 180, ensures that excess of agent probing molecule 122 will not reach the substrate molecule 152 deposited on the “substrate line” 180.

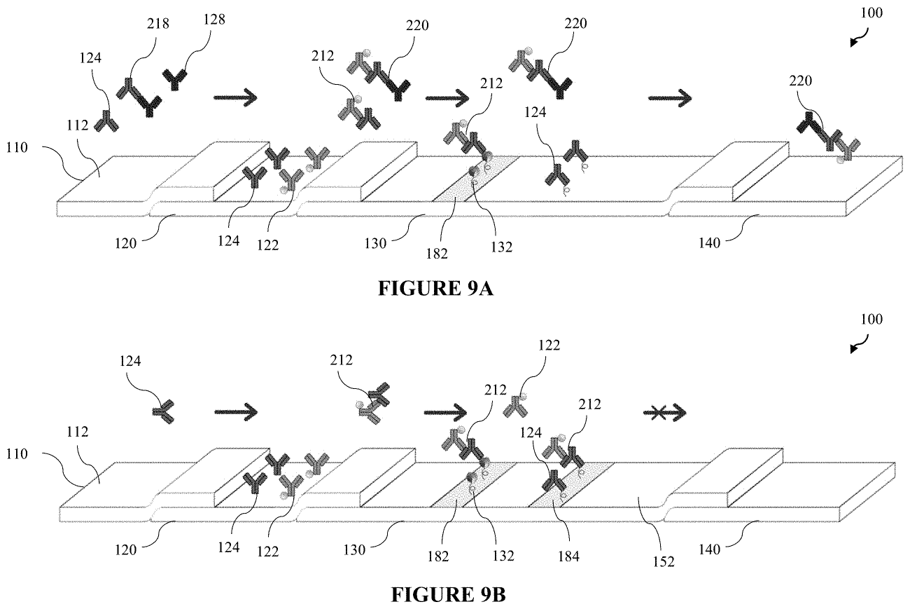

Reference is now made to FIG. 9, which is a simplified illustration of how a device 100 works during an assay measurement according to some embodiments of the present invention, in the case of a sample comprising an analyte. In some embodiments, the device comprises a section 1 110 with a sample collecting surface 112, a section 2 120 comprising a surface with an agent 124 and an agent probing molecule 122 linked to a reporter molecule, a section 3 130 comprising surface functionalized with a target molecule 132, and an agent 124, and a section 4 140.

Reference is now made to FIG. 9A. In some embodiments, a liquid sample comprising an analyte comprising a neutralizing anti-agent antibody 128 , and/or a complex 218 comprising the agent 124 bound or in contact with the neutralizing anti-agent antibody 128, is deposited in section 1 110. The sample migrates to section 2 120, via lateral flow, where it encounters the agent 124 and the agent probing molecule 122. A complex 220 is formed based on molecular recognition (such as affinity-based interaction or binding, as described herein), wherein complex 220 comprises the agent 124 bound or in contact with the agent probing molecule 122, and with the neutralizing anti-agent antibody 128, and wherein the agent probing molecule 122 is bound to a reporter molecule generating a trigger. A complex 212 comprising the agent 124 bound or in contact with the agent probing molecule 122 may/is also formed. The complex formed 220, continues to migrate via lateral flow to section 3 130 comprising the target molecule 132. The complex formed 220 will not interact with the target molecule 132 and will continue to migrate through section 3 130 to section 4 140. The complex 212 comprising the agent 124 bound or in contact with the agent probing molecule 122 will interact or bind to the target molecule 132 and therefore immobilized to section 3 130 on the “capture line” 182. Upon supplementation of a substrate molecule, such as described herein, the complex formed 212 will interact with the substrate molecule and a signal will be generated on the “capture line” 182, thereby confirming the presence of a non-neutralized agent, e.g., the drug antibody 124 in the sample. Further, complex 220 that migrated to section 4 140 will also interact with the substrate molecule, thereby generating a signal on the surface of section 4 140. The type of signal generation will depend on the reporter molecule used that is conjugated to the reporter molecule and the substrate molecule supplemented thereto.

Reference is now made to FIG. 9B. In some embodiments, a liquid sample comprising an analyte devoid of a neutralizing anti-agent antibody 128 , and/or a complex 218 comprising the agent 124 bound or in contact with the neutralizing anti-agent antibody 128, is deposited in section 1 110. The sample migrates to section 2 120, via lateral flow, where it encounters the agent 124 and the agent probing molecule 122. A complex 212 comprising the agent 124 bound or in contact with the agent probing molecule 122 is formed. The complex 212 comprising the agent 124 bound or in contact with the agent probing molecule 122 will migrate to section 3 130 where it will interact or bind to the target molecule 132 and, therefore immobilized thereto. Further, a complex 212 will also form where the surface of section 3 130 is functionalized with the agent 124. Upon supplementation of a substrate molecule, such as described herein, the complexes formed 212 will interact with the substrate molecule and a signal will be generated on the “capture lines” 182 and 184, thereby confirming the presence of a non-neutralized agent, e.g., the drug antibody 124 in the sample, and the absence of a neutralizing anti-agent antibody 128. The type of signal generation will depend on the reporter molecule used that is conjugated to the reporter molecule and the substrate molecule supplemented thereto.

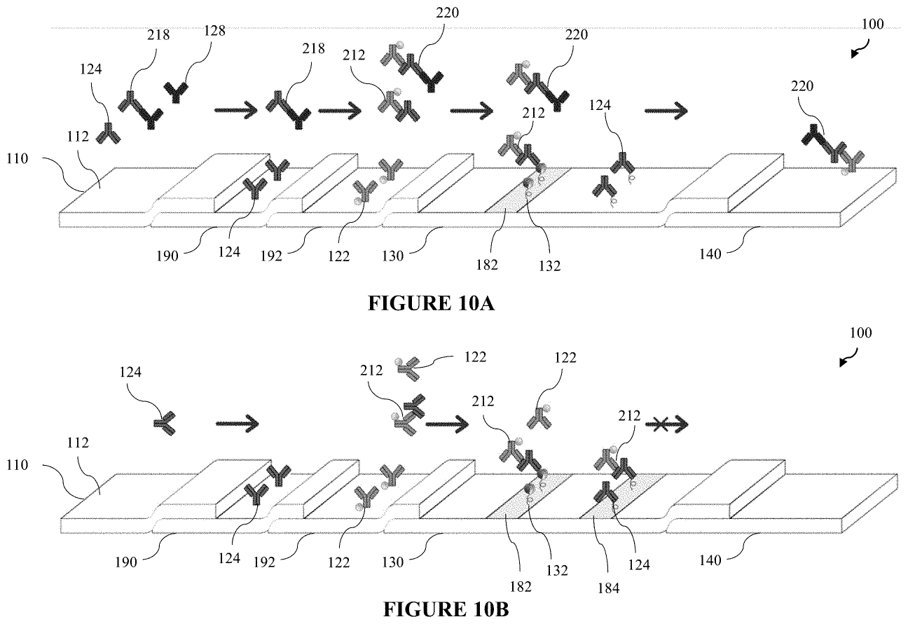

Reference is now made to FIG. 10, which is a simplified illustration of how a device 100 works during an assay measurement according to some embodiments of the present invention, in the case of a sample comprising an analyte. In some embodiments, the device comprises a section 1 110 with a sample collecting surface 112, a first section 2 190 comprising a surface with an agent 124, a second surface 2 192 comprising an agent probing molecule 122 linked to a reporter molecule, a section 3 130 comprising surface functionalized with a target molecule 132, and an agent 124, and a section 4 140.

Reference is now made to FIG. 10A. In some embodiments, a liquid sample comprising an analyte comprising a neutralizing anti-agent antibody 128 , and/or a complex 218 comprising the agent 124 bound or in contact with the neutralizing anti-agent antibody 128, is deposited in section 1 110. The sample migrates to the first section 2 190, via lateral flow, where it encounters the agent 124 and thereafter by lateral flow to the second section 2 192 comprising the agent probing molecule 122. A complex 220 is formed based on molecular recognition (such as affinity-based interaction or binding, as described herein), wherein complex 220 comprises the agent 124 bound or in contact with the agent probing molecule 122, and with the neutralizing anti-agent antibody 128, and wherein the agent probing molecule 122 is bound to a reporter molecule generating a trigger. A complex 212 comprising the agent 124 bound or in contact with the agent probing molecule 122 may/is also formed. The complex formed 220, continues to migrate via lateral flow to section 3 130 comprising the target molecule 132. The complex formed 220 will not interact with the target molecule 132 and will continue to migrate through section 3 130 to section 4 140. The complex 212 comprising the agent 124 bound or in contact with the agent probing molecule 122 will interact or bind to the target molecule 132 and therefore immobilized to section 3 130 on the “capture line” 182. Upon supplementation of a substrate molecule, such as described herein, the complex formed 212 will interact with the substrate molecule and a signal will be generated on the “capture line” 182, thereby confirming the presence of a non-neutralized agent, e.g., the drug antibody 124 in the sample. Further, complex 220 that migrated to section 4 140 will also interact with the substrate molecule, thereby generating a signal on the surface of section 4 140. The type of signal generation will depend on the reporter molecule used that is conjugated to the reporter molecule and the substrate molecule supplemented thereto.

Reference is now made to FIG. 10B. In some embodiments, a liquid sample comprising an analyte devoid of a neutralizing anti-agent antibody 128 , and/or a complex 218 comprising the agent 124 bound or in contact with the neutralizing anti-agent antibody 128, is deposited in section 1 110. The sample migrates to the first section 2 190, via lateral flow, where it encounters the agent 124 and thereafter via lateral flow to the second section 2 192 comprising the agent probing molecule 122. A complex 212 comprising the agent 124 bound or in contact with the agent probing molecule 122 is formed. The complex 212 comprising the agent 124 bound or in contact with the agent probing molecule 122 will migrate to section 3 130 where it will interact or bind to the target molecule 132 and, therefore immobilized thereto. Further, a complex 212 will also form where the surface of section 3 130 is functionalized with the agent 124. Upon supplementation of a substrate molecule, such as described herein, the complexes formed 212 will interact with the substrate molecule and a signal will be generated on the “capture lines” 182 and 184, thereby confirming the presence of a non-neutralized agent, e.g., the drug antibody 124 in the sample, and the absence of a neutralizing anti-agent antibody 128. The type of signal generation will depend on the reporter molecule used that is conjugated to the reporter molecule and the substrate molecule supplemented thereto.

### Section 1

In some embodiments, a device as described herein comprises a section 1, comprising a sample collecting surface.

In some embodiments, a collecting surface is a filter. In some embodiments, a collecting surface is a solid support that may hold the sample. In some embodiments, a collecting surface comprises a membrane or matrix, wherein the membrane or matrix is as described hereinabove.

In some embodiments, a collecting surface comprises a material capable of absorbing or adsorbing a liquid sample.

In some embodiments, the size and shape of section 1 is not critical, and it may vary.

In some embodiments, the sample collecting surface is comprised of filter for whole cell and large bodies filtration.

In some embodiments, the sample collecting surface is comprised of a material allowing protein-protein interactions to take place, such as cellulose or nitrocellulose, PVA, and others as described herein.

In some embodiments, the sample collecting surface contains a buffer for controlling pH and ionic strength.

As used herein the term “sample collecting surface” refers to a surface wherein the sample is applied. The applied sample migrates sequentially from the sample collecting surface in section 1 to section 2, section 3, section 4, and section 5, in this specific order.

As used herein, the terms “applied”, “loaded”, and “deposited”, are interchangeable.

### Section 2

In some embodiments, a device as described herein comprises at least one section 2, comprising a surface comprising an agent, and an agent probing molecule linked or bound to a reporter molecule. In some embodiments, section 2, comprises a surface comprising a deposited agent and a probing molecule linked to a reporter molecule. In some embodiments, the at least one section 2 comprises two sections 2. In some embodiments, the first section 2 comprises a surface comprising an agent. In some embodiments, the second section 2 comprises an agent probing molecule linked or bound to a reporter molecule.

In some embodiments, the agent comprises a drug. In some embodiments, the agent is a drug. In some embodiments, the drug is affecting a target molecule. In some embodiments, affecting is modulating. In some embodiments, modulating is increasing or decreasing. In some embodiments, the drug reduces or inhibits the activity of the target molecule. In some embodiments, the drug reduces or inhibits the signaling of the target molecule. In some embodiments, the drug reduces or inhibits the signaling and the activity of the target molecule.

In some embodiments, the agent is an antigen binding molecule. In some embodiments, the agent is an antibody. In some embodiments, the agent is an aptamer. In some embodiments, the agent is an artificial entity. In some embodiments, the agent is a chimera. In some embodiments, the agent is any one of: Infliximab, adalimumab, certolizumab, and golimumab.

In some embodiments, the agent is any one of: Nivolumab, Ipilimumab, Pembrolizumab, Cemiplimab, Atezolizumab, Avelumab, Durvalumab, Bevacizumab, Cetuximab, Panitumumab, Rituximab, Alemtuzumab, Trastuzumab, Ibritumomab, Lambrolizumab, Tremelimumab (formerly Ticilimumab), and Ado-Trastuzumab emtansine.

In some embodiments, the chimera comprises a carrying molecule and antigen binding molecule attached or linked thereto. In some embodiments, linked is directly linked or indirectly linked, such as via a linker. In some embodiments, the linker is a flexible or a rigid linker. In some embodiments, the carrying molecule is a protein. Non-limiting examples of carrying proteins include, but are not limited to, bovine serum albumin (BSA), human thyroglobulin (hTg) peptide, or others. In some embodiments, the antigen binding molecule is characterized by having specific binding affinity to the target molecule as describe herein. In some embodiments, the antigen binding molecule is a portion of an antibody. In some embodiments, the antigen binding molecule is a portion of the Fab domain of an antibody. In some embodiments, the agent probing molecule, as disclosed hereinbelow, has specific affinity to antigen binding molecule. In some embodiments, the chimera comprises a first region capable of binding the target molecule, and a second region being recognized by the immune system. In some embodiments, the first region comprises a binding counterpart of the target molecule. In some embodiments, the first region comprises a receptor or a binding domain thereof, capable of binding the target molecule. In some embodiments, the second region comprises an IgG Fc fragment.

In some embodiments the agent probing molecule has specific affinity to the agent. In some embodiments, the reporter molecule generates a chemically and/or an electric and/or a magnetic, and/or a piezoelectric, and/or a fluorescent and/or a physically detectable reaction or signal. In some embodiments, the reporter molecule generates a trigger. In some embodiments, the agent probing molecule is dried on the surface of section 2. In some embodiments, the recognition molecule is unbound to the surface of section 2. In some embodiments, the trigger induces a signal formation upon contacting a substrate molecule. In some embodiments, the trigger is capable to interact chemically (e.g. via a reaction and/or a non-covalent binding), physically (e.g. via photon-induced excitation), via interactions with ionizing radiation, or by inducing electromagnetic field-based interaction. In some embodiments, the trigger comprises at least one of: a reactive compound (such as a peroxide, or any compound capable of reacting with the substrate molecule so as to generate a signal), an electromagnetic radiation, an ionizing radiation, and a charged particle or a combination thereof. In some embodiments, the trigger is a photon having a wavelength sufficient to induce a fluorescence, a luminescence, electrochemiluminescence, a phosphorescence or a colorimetric reaction of the substrate molecule.

The term “agent probing molecule” as used herein refers to a molecule possessing a high affinity to (e.g., an equilibrium dissociation constant values of K_{d}≤10⁻⁹ M), in a biologically relevant system (e.g., in vitro, ex vivo or in vivo). In some embodiments, the “agent probing molecule” comprises a “reporter molecule”. In some embodiments, the “agent probing molecule” comprises a “reporter molecule” which is capable of generating a measurable signal detectable by external means.

In some embodiments, the agent probing molecule is an antibody. In some embodiments, the agent probing molecule is an aptamer. In some embodiments, the agent probing molecule is an artificial entity. In some embodiments, the agent probing molecule is a chimera.

The term “reporter molecule” as used herein refers to a chemical group or a molecular motif possessing medium to high affinity towards a molecular reagent or a biomolecule that induces or mediates a reaction that yields a product, that can be monitored instrumentally. In some embodiments, “reporter molecule” include chromogens, catalysts such as enzymes, luminescent compounds such as fluorescein and rhodamine, chemiluminescent compounds such as dioxetanes, acridiniums, phenanthridinium and luminol, radioactive elements, electroactive compounds, TEMPO, 1,4,5,8-naphthalenetetracarboxylic diimide (NTCDI), stilbene, upconversion particles, and direct visual labels. The selection of a particular reporter molecule is not critical, but it will be capable of producing a signal either by itself or in interaction with one or more additional substances.

Examples of reporter enzymes which can be used to practice the invention include peroxidases, hydrolases, lyases, oxidoreductases, transferases, isomerases, phosphatases, and ligases. Further non-limiting examples of reporter enzymes include glucose oxidase, phosphatases, esterases, glycosidases and peroxidases. In some embodiments, the reporter molecule is a protein, an enzyme, a horseradish peroxidase (HRP), a nucleotide, a dye, a quantum dot, a fluorophore, a dendrimer, a gold particle, a silver particle, or a platinum particle. In some embodiments, the reporter molecule generates a chemically active trigger such as hydrogen peroxide, which oxidizes the substrate molecule.

In some embodiments, a reporter molecule is selected from: an enzyme, a luminescent substrate compound, a fluorophore, electrochemical active compound, fluorophores (organic, quantum dots, fluorescent proteins), organic dye, magnetic particles, gold particles.

### Section 3

In some embodiments, a device as described herein comprises at least one section 3 comprising a surface functionalized with a target molecule and an agent, wherein the surface is as described hereinabove. In some embodiments, the target molecule is bound to the surface of section 3. In some embodiments, the at least one section 3 comprises two sections 3. In some embodiments, the first section 3 comprises a surface functionalized with a target molecule. In some embodiments, the second section 3 comprises a surface functionalized with an agent, as disclosed herein.

In some embodiments, the device comprises at least one first section 3. In some embodiments, the device comprises a plurality of the first section 3. In some embodiments, the device comprises at least one second section 3. In some embodiments, the device comprises a plurality of the second section 3.

As used herein, the term “plurality” comprises any integer equal to or greater than 2.

In some embodiments, the entire surface of the at least one second section 3 is functionalized with an agent as disclosed herein. In some embodiments, an agent as disclosed herein at least partially functionalizes or covers the surface of the at least one second section 3.

In some embodiments, an agent as disclosed herein functionalizes or covers a plurality of subsections of the surface of the at least one second section 3.

In some embodiments, the target molecule is a peptide.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, the target is selected from: a cytokine, a chemokine, an integrin, an adhesion molecule, and an immune checkpoint molecule.

In some embodiments, the target comprises any endogenous molecule involved, initiates, propagates, enhances, taking part in the pathogenesis and/or pathophysiology.

As used herein, the term “cytokine” encompasses any small immunomodulating peptide of ˜5-20 kDa.

In some embodiments, a cytokine is selected from: tumor necrosis factor (TNF), interleukin (IL), a chemokine, and interferon. In one embodiment, TNF is TNF alpha. In one embodiment, interferon is interferon gamma. In some embodiments, the cytokine is selected from: IL-6, IL-6 receptor, IL-2, IL-22, IL-7, IL-12, p40 subunit, and IL-23 p19 subunit.

As used herein, the term “chemokine” encompasses any small any cytokine and/or a signaling protein which is secreted by a cell and is capable of inducing chemotaxis of a neighboring cell.

In one embodiment, the chemokine is any one of: CCL3, CCL26, and CXCL7.

As used herein, the term “integrin” encompasses any transmembrane protein, e.g., a receptor, capable of promoting adhesion of cell-extracellular matrix.

In some embodiments, the integrin is selected from: a4b7 integrin, b7 integrin, aE integrin, and a4 integrin.

Types of cytokines are well known in the art inclusive of methods for their identification and/or quantification.

As used herein, the term “immune checkpoint” encompasses any regulator of the immune system or actions thereof, taken so as to inhibit, control, or prevent the immune system from attacking the host's cells indiscriminately.

In some embodiments, an immune checkpoint is selected from: programed death ligand 1 (PD-L1), programed death protein 1 (PD-1), or cytotoxic T-lymphocyte-associated protein 4 (CTLA-4).

In some embodiments, if a sample without an analyte (e.g., a neutralizing anti-drug antibody) is used, during the migration, of the sample, the excess of free agent probing molecule will be conjugated into the section 3 functionalized with the agent (such as any one of the “capture lines” 182, 184, and both, as disclosed herein) and will not migrate further to section 4.

In some embodiments, if a sample comprising an analyte (e.g., a neutralizing anti-drug antibody) is used, during the migration, of the sample, the excess of free agent probing molecule will be conjugated into the section 3 functionalized with the agent (such as the “capture line” 182, as disclosed herein) and will not migrate further to section 4.

In some embodiments, if a sample without an analyte comprising an anti-drug neutralizing antibody is used, during the migration, of the sample, the excess of free agent probing molecule will be conjugated into the section 3 functionalized with the agent (such as any one of the “capture lines” 182, 184, and both, as disclosed herein) and will not migrate further to section 4.

In some embodiments, if a sample with an analyte is used, since the analyte-agent probing molecule-reporter molecule complex is formed before section 3, the sample will continue and migrate to section 4 or 5 comprising a surface with a deposited substrate molecule, thereby generating a signal. The type of signal generation will depend on the reporter molecule used that is conjugated to the recognition molecule and/or the substrate molecule deposited. In some embodiments, the section 4 or 5 is devoid of a substrate molecule.

In some embodiments, if a sample with an analyte comprising an anti-drug neutralizing antibody is used, since the anti-drug neutralizing antibody-agent-agent probing molecule-reporter molecule complex is formed before section 3, the sample will continue and migrate to section 4 or 5 comprising a surface either: (i) with a deposited substrate molecule, thereby generating a signal; or (ii) being devoid of a substrate molecule. The type of signal generation will depend on the reporter molecule used that is conjugated to the recognition molecule and/or the substrate molecule deposited.

In some embodiments, an equivalent to the analyte is used. In some embodiments, an equivalent to the analyte refers to an analogous molecule. An equivalent to the analyte is a molecule with interaction to the same active site on the agent. In some embodiments, an analyte analog can be a synthetic peptide or a subunit of a protein.

### Section 4

In some embodiments, a device as described herein comprises a section 4. In some embodiments, section 4 comprises a non-functionalized surface.

In some embodiments, a device as described herein comprises a section 4 comprising a surface functionalized with an agent, wherein the surface is as described hereinabove. In some embodiments, the agent is bound to the surface of section 4.

In some embodiments, if a sample without an analyte is used, during the migration, of the sample, the excess of free agent probing molecule will be conjugated into the section 4 functionalized with the agent and will not migrate further to section 5.

In some embodiments, if a sample comprising an analyte is used, during the migration, of the sample, the excess of free agent probing molecule will be conjugated into the section 4 functionalized with the agent and will not migrate further to section 5.

In some embodiments, if a sample without an analyte comprising an anti-drug neutralizing antibody is used, during the migration, of the sample, the excess of free agent probing molecule will be conjugated into the section 4 functionalized with the agent and will not migrate further to section 5.

In some embodiments, if a sample with an analyte is used, since the analyte-agent probing molecule-reporter molecule complex is formed before section 3, the sample will continue and migrate to section 5 comprising a surface with a deposited substrate molecule, thereby generating a signal. The type of signal generation will depend on the reporter molecule used that is conjugated to the recognition molecule and/or the substrate molecule deposited.

### Section 5

In some embodiments, a device according to the present invention, comprises a section 5 comprising a surface in contact or bound to a substrate molecule, wherein the surface is as described hereinabove.

In some embodiments, section 5 comprises a surface comprising an electrode. In some embodiments, section 5 comprises a surface in contact with or bound to a substrate molecule selected from a fluorophore, a luminophore, a photo-luminophore, a radio-luminescent material, and a light-reactive material or a combination thereof. In some embodiments, the substrate molecule comprises a molecule capable of reacting with peroxide, so as to form a detectable signal.

In some embodiments, when the substrate molecule of section 5 encounters a reporter molecule it emits a signal with a certain intensity. In some embodiments, the signal intensity is compared to a calibration curve or an indicative value. In some embodiments, the signal obtained is proportional to the analyte concentration in the sample. In some embodiments, the signal obtained is proportional to the analyte concentration in the sample and the time from the sample reaching section 5 to the time of measurement. In some embodiments, the signal obtained is proportional to the neutralizing anti-drug antibody concentration in the sample. In some embodiments, the signal obtained is proportional to the neutralizing anti-drug antibody concentration in the sample and the time from the sample reaching section 5 to the time of measurement.

In some embodiments, the substrate molecule is a colorimetric agent. In some embodiments, the substrate molecule is capable of reacting with the trigger (such as a peroxide) to result in color change. In some embodiments, the substrate molecule is a color producing substrate molecule such as 5-Bromo-4-Chloro-3-IndolylPhosphate (BCIP) or 3,3′,5,5′-tetramethylbenzidine (TMB), 4-CN DAB, chromogenic.

In some embodiments, the type of signal depends on the chosen reporter molecule and/or substrate molecule.

In some embodiments, signal detection, quantification or both is done using a reader or detection unit. In some embodiments, the device of the invention further comprises a detection unit. In some embodiments, the detection unit is in operable communication with the device. In some embodiments, the detection unit is in operable communication with section 5. In some embodiments, the detection unit is configured to detect the signal generated by the substrate molecule. In some embodiments, the detection unit comprises electric circuitry.

### Calibration Area

In some embodiments, a device according to the present invention further comprises a calibration area.

In some embodiments, a device according to the present invention further comprises a calibration area positioned between section 2 and section 3 and comprising a substrate molecule. In some embodiments, calibration area is in fluid communication with or is coupled to section 2 and section 3.

In some embodiments, a device as described herein comprises calibration area comprising a substrate molecule, wherein the calibration area is placed adjacent to section 2. In some embodiments, a device as described herein comprises calibration area comprising a substrate molecule placed between section 2 and section 3. In some embodiments, the calibration area comprises a membrane, wherein the membrane is as described herein.

In some embodiments, the calibration area is placed before the surface functionalized with the target molecule. In some embodiments, the calibration area is devoid of the target molecule. In some embodiments, the calibration area is devoid of an agent probing molecule. In some embodiments, the calibration area is devoid of a reporter molecule. In some embodiments, when the substrate molecule of the calibration area encounters a reporter molecule, the reporter molecule generates a trigger, that upon interaction with the substrate molecule generates a signal giving an indication for the functionality and quantity of the reporter molecule and a reference of total signal intensity. In some embodiments, the signal intensity is used for signal calibration.

As used herein, the term “detection unit” refers to an instrument capable of detecting and/or quantitating data, such as on the sections described herein. The data may be visible to the naked eye but does not need to be visible. In some embodiments, the detection unit is in operable communication with a processor. A processor is of a general-purpose computer, special purpose computer, or other programmable data processing apparatus to produce a machine, such that the instructions, which execute via the processor of the computer or other programmable data processing apparatus, create means for implementing the functions of the device. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the device. In some embodiments, the signal received form the device is processed by a software so as to generate an output, such as a positive or a negative reporting. In some embodiments, analysis the data generated by the device of the invention and/or the method or assay using the device comprises the use of cloud analytics. In some embodiments, analysis the data generated by the device of the invention and/or the method or assay using the device comprises the use of artificial intelligence (AI).

In some embodiments, the program code is excusable by a hardware processor.

In some embodiments, the hardware processor is a part of the control unit.

In some embodiments, there is further provided a read-out of the assay carried out in the device may be detected or measured using any suitable detection or measuring means known in the art. The detection means may vary depending on the nature of the read-out of the assay. In some embodiments, disclosed device also relates to an apparatus including the device in any embodiments thereof, and a detection unit as described herein.

In some embodiments, the detection unit provides a positive reporting. In some embodiments, the detection unit provides a negative reporting. As used herein “positive reporting” refers to an increase in the detection signal with the increase of analyte concentration. As used herein the term “negative reporting” refers to no detection signal.

In some embodiments, a reader is an electrochemical detection unit. In some embodiments, a reader is an electro-chemiluminescent detection unit. In some embodiments, a reader is a colorimetric detection unit. In some embodiments, a detection unit comprises a photodetector such as Photomultiplier Tubes (PMTS), CCD camera or complementary MOS (CMOS). In some embodiments, a detection unit is a cellphone. In some embodiments, a detection unit will include light source for excitation of a fluorescent reporter molecule and a photo detector. In some embodiments, a detection unit is a human.

In some embodiments, a signal is a color change. In some embodiments, a signal is light generation. In some embodiments, a signal is an electron flow. In some embodiments, a signal is an excited light source.

As used herein, the term “color” refers to the relative energy distribution of electromagnetic radiation within the visible spectrum. Color can be assessed visually or by using equipment, such as a photosensitive detector.

As used herein, the term “color change” refers to a change in intensity or hue of color or may be the appearance of color where no color existed or the disappearance of color.

In some embodiments, section 5 further comprises an active-pixel sensor (APS) or an electrode.

In some embodiments, the device further comprises diffusible membranes located between the sections of the device, which modulate sample flow rate and interaction time between reagents during measurement procedure.

In some embodiments, a diffusible membrane is made of Polyvinyl alcohol (PVA), paraffin, but is not limited to this preferred material.

In some embodiments, the device will be introduced to vibrations with frequency ranging between 0.1 kHz and 1000 kHz, the vibration will encourage interactions between reagents and increase efficiency. In some embodiments, the vibrations are originating from an internal section. In some embodiments, the vibrations are originating from an external device.

In some embodiments, the flow can be modulated using a magnetic field.

In some embodiments, a device according to the present invention, is capable of detecting lower amounts of an analyte, e.g., comprising a neutralizing anti-drug antibody, in a sample when compared to a typical enzyme-linked immunosorbent assay (ELISA).

In some embodiments, a device according to the present invention detects the presence of an analyte in a solution with a concentration lower than 25 ng/mL. In some embodiments, a device according to the present invention detects the presence of an analyte in a solution with a concentration lower than 25 ng/mL, lower than 24 ng/mL, lower than 20 ng/mL, lower than 15 ng/mL, lower than 10 ng/mL, lower than 8 ng/mL, lower than 7 ng/mL, or lower than 5 ng/mL, including any value therebetween.

In some embodiments, the mole to mole (m:m) ratio of a reporter molecule in section 2 and a substrate molecule in section 5 is in the range of 1:1 to 1:1,000. In some embodiments, the m:m ratio of a reporter molecule in section 2 and a substrate molecule in section 5 is in the range of 1:1 to 1:900, 1:1 to 1:700, 1:1 to 1:500, 1:1 to 1:200, 1:1 to 1:100, 1:1 to 1:50, 1:1 to 1:25, or 1:1 to 1:10, or any value and range therebetween. Each possibility represents a separate embodiment of the invention.

### The Method

According to some embodiments, there is provided a method for determining the presence of an analyte in a sample, comprising the steps of: contacting section 1 of the device of the invention with a sample; and detecting the presence of a signal, wherein the presence of the signal is indicative of the presence of the analyte in the sample, thereby determining the presence of the analyte in the sample.

In some embodiments, the method comprises contacting the device with an effective amount of a substrate molecule as disclosed herein. In some embodiments, the method comprises contacting the device with the substrate after the sample has been contacted or loaded to the device. In some embodiments, the method comprises contacting the device with the substrate after the sample has been contacted or loaded to the device and after the sample has been allowed to migrate through all sections of the device as disclosed herein. In some embodiments, the method comprises contacting the device with an effective amount of a substrate after the sample or any component, fraction, or portion thereof, has migrated to section 4 or 5, as disclosed herein. In some embodiments, the method comprises contacting the device with an effective amount of a substrate after the sample or any component, fraction, or portion thereof, has migrated to section 4, as disclosed herein. In some embodiments, the method comprises contacting the device with an effective amount of a substrate after the sample or any component, fraction, or portion thereof, has migrated to section 5, as disclosed herein.

In some embodiments, determining the presence of the analyte is indicative of the presence of an anti-drug neutralizing antibody in the sample or the subject.

In some embodiments, determining the presence of the analyte is indicative of the presence of drug antibody in the sample or the subject.

In some embodiments, the analyte comprises or is a drug, such as, but not limited to an antibody drug.

In some embodiments, the analyte comprises or is an anti-drug neutralizing antibody.

In some embodiments, the method further comprises a step of quantifying the amount of the analyte in a sample, comprising: determining the amount of the signal, and comparing it to a calibration curve or an indicative value, thereby quantifying the amount of the analyte in the sample.

In some embodiments, the method further comprises a step of determining the amount of a drug in the sample. In some embodiments, determining the amount of a drug comprises contacting a second device, e.g., other than the device of the invention. In some embodiments, the second device comprises a section 1, a section 2, a section 3 and a section 4, wherein section 2 is coupled to section 1; section 3 is coupled to section 2 and to section 4, section 3 comprises a surface functionalized with the drug; sections 1 to 4 are arranged along a horizontal axis and in liquid communication allowing lateral flow of liquid from section 1 through section 2 and section 3 to section 4. In some embodiments, the second device comprises a section 1 comprising a sample collecting surface; section 2 comprising a surface deposited with a recognition molecule having specific affinity to analyte linked to a reported molecule, wherein the reporter molecule generates a chemically and/or electrically and/or a physically detectable reaction; section 4 comprising a surface deposited with a substrate; and section 5 comprising a surface available for holding excess sample.

In one embodiment, the second device further comprises a calibration area comprising a substrate placed between section 2 and section 3. In one embodiment, the sample diffuses from section 1 to section 5 of the second device.

In some embodiments, the second device comprises or consists of the device disclosed in International Patent Application No. PCT/IL2019/051446, which is incorporated herein by reference in its entirety.

In some embodiments, a device according to the present invention further comprises a calibration area. In some embodiments, a device according to the present invention further comprises a calibration area disposed between section 2 and section 3 and wherein the calibration area is in contact with the substrate molecule. In some embodiments, the substrate molecule of section 5 and the substrate molecule of the calibration area are identical.

The term “contacting”, as used herein, refers generally to providing excess of one component, reagent, analyte, or sample to another.

In some embodiments, a sample is deposited, applied, or loaded on section 1, and flows sequentially from section 1 to section 4 or 5.

In some embodiments, detecting the presence of a signal is in section 4 or 5. In some embodiments, if an analyte is present in a sample, a signal will be detected in section 4 or 5. In some embodiments, if no analyte is present in a sample, a signal will not be detected in section 4 or 5.

In some embodiments, detecting the presence of a signal is in sections 3 and 4. In some embodiments, if an analyte is present in a sample, a signal will be detected in section 3 (e.g., on the “capture line” 182 as disclosed herein) and 4. In some embodiments, if an analyte is absent from a sample, a signal will be detected in section 3 (e.g., on the “capture lines” 182, 184, or both as disclosed herein).

Specifications of section 1, section 2, section 3, section 4, and section 5 and detection of a signal are described elsewhere herein.

In some embodiments, the sample diffuses from section 1 to section 4 or 5. In some embodiments, all dissolved or dispersed components of the sample diffuse at substantially equal rates and with relatively unimpaired flow laterally from section 1 to section 4 or 5.

In some embodiments, there is provided a method for diagnosing or determining drug desensitization or intolerance.

In some embodiments, the presence of a signal can be detected within 1 min to 40 min after applying a sample in section 1. In some embodiments, the presence of a signal can be detected within: 1 min to 30 min, 1 min to 20 min, 1 min to 15 min, 1 min to 10 min, 2 min to 30 min, 5 min to 30 min, 5 min to 20 min, 5 min to 15 min, or 5 min to 10 min, after applying a sample in section 1, or any value and range therebetween. Each possibility represents a separate embodiment of the invention.

### The Kit

According to some embodiments, there is provided a kit comprising: (a) a first device, comprising the device of the invention; and (b) a second device comprising: a section 1, a section 2, a section 3 and a section 4, wherein: section 2 is coupled to section 1; section 3 is coupled to section 2 and to section 4, section 3 comprises a surface functionalized with an agent; and sections 1 to 4 are arranged along a horizontal axis and in liquid communication allowing lateral flow of a liquid sequentially from sections 1 to 4.

In some embodiments, the kit is for determining the presence, amount, or both, of any one of: the agent, an antibody neutralizing the agent, or both, in a sample.

According to some embodiments, there is provided a kit, comprising: at least a section 1 comprising a sample collecting surface; a section 2, comprising a surface deposited with an agent having specific binding affinity to a target molecule and an agent probing molecule having specific affinity to the agent and linked to a reporter molecule, wherein the reporter molecule generates a chemically and/or electrically and/or a physically detectable reaction; a section 3 functionalized with the target; a section 4 comprising a surface functionalized with the agent, and a section 5 comprising a surface deposited with a substrate molecule.

In some embodiments the kit, comprises: a section 1, a section 2, a section 3, and a section 4. In some embodiments, the kit further comprises a section 5. In some embodiments, the kit comprises an agent. In some embodiments, the kit comprises an agent probing molecule comprising a reporter molecule. In some embodiments, the kit comprises a substrate molecule.

In some embodiments, the kit further comprises instructions for depositing section 2 with the agent and/or agent probing molecule. In some embodiments, the kit further comprises instructions for depositing section 4 of the second device with a substrate molecule. In some embodiments, the kit further comprises instructions for depositing section 5 of the device of the invention with a substrate molecule.

In some embodiments, a kit according to the present invention comprises instructions for connecting a section 1, a section 2, a section 3, a section 4, and a section 5 in an axial and consecutive order and/or partially overlapping.

In some embodiments, the kit further comprises a sample collecting instrument. In some embodiments, a sample collecting instrument is a graduated measuring instrument. In some embodiments, a sample collecting instruments is used to collect a sample applying the sample in the sample collecting surface. Non-limiting examples of collecting instruments that can be used according to the present invention include swab, wooden spatula, pipette, or any other suitable form of a sample collecting apparatus.

In some embodiments, the kit comprises at least two sections 5, wherein each one of the different sections 5 comprises a different substrate molecule. In some embodiments, at least one of the substrates comprises an active-pixel sensor (APS) or an electrode.

According to some embodiments of the invention, there is provided kit for determining the presence of an analyte in a sample.

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments unless the embodiment is inoperative without those elements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

A 1:20 diluted pooled negative sera were spiked with 0, 50, 200, 350, 500, and 650 ng/ml of neutralizing anti Infliximab (IFX) antibody (HCA233, BIO-RAD) and mixed with IFX at a concentration of 30 ng/ml and HRP-labeled anti-IFX non-neutralizing antibody (HCA216P, BIO-RAD) at a concentration of 45 ng/ml, representing the mixture of entities created after the sample passes the conjugate membrane. The mixed samples were passed through a tumor necrosis factor alpha (TNFα) capture membrane followed by an IFX capture membrane and reached the substrate membrane. The signal generated by the oxidized substrate was quantified using CCD camera photodetector (EXi Blue Qimaging).

### Example 1

### Neutralizing-antibodies Detection Assay

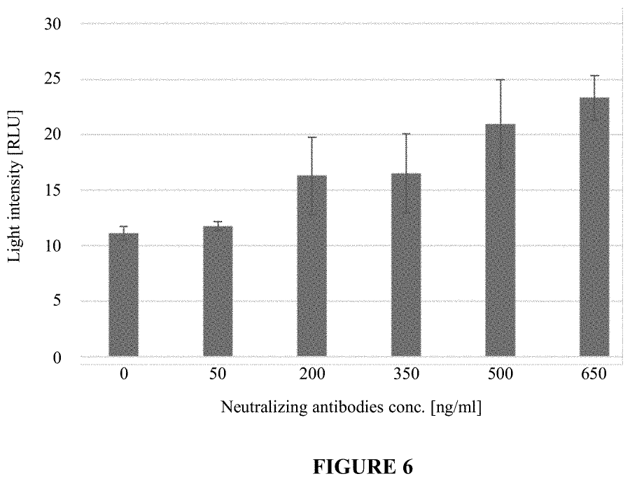

The assay's rationale was tested with naïve serum samples spiked with a commercial neutralizing antibody. As shown in FIG. 6, the assay displayed a dose dependent response to a range of neutralizing antibody concentrations, with a cut-off signal when the samples contained neutralizing ADAs at a concentration, as low as, 200 ng/ml. These tests had validated the assay's strategy.

Further steps were developed to fit the clinical range of IFX levels found in patients' sera and the predicted levels of neutralizing antibodies. These optimization steps include testing of different concentrations of both the capture reagents, confirming the depletion of all excess free IFX and free HRP-labeled anti-IFX, as well as the concentration of the IFX added to saturate all the free neutralizing antibodies. The assay susceptibility to nonspecific matrix effects is thoroughly checked. To determine the assay cut point, a panel of separate serum samples of mixed gender individuals not exposed to the drug are tested by two operators.

### Example 2

### Substrate Line Assay

The test was performed as a deep test assay. First, the inventors dried anti-drug-Ab-HRP in concentration of 4 μg/mL in Tris saline 0.05 M with 0.5% BSA and 2% Lactose for 1.5 hours. Three capture lines of drug (also referred to herein as “agent) and one capture line of a target molecule (such as TNFα) all of them in concentration of 0.6 mg/mL in PBS saline were deposited onto the membrane with a substrate line containing 3 mg/mL of DAB and 2 mg/mL of 4-CN in Tris base 0.05M with 40% methanol. The running buffer used was Tris base 0.05 M+1% PEG 20K+27.2 mg/mL Imidazole+0.1% H₂O₂. As a sample the inventors used spiked porcine serum with 50 μg/mL of drug and increasing concentrations of commercial neutralizing ADA (nADA). The inventors let the sample and running buffer to run for 20 minutes and took pictures during the test. After the time was completed, the inventors added DAB substrate onto the membrane and took pictures.

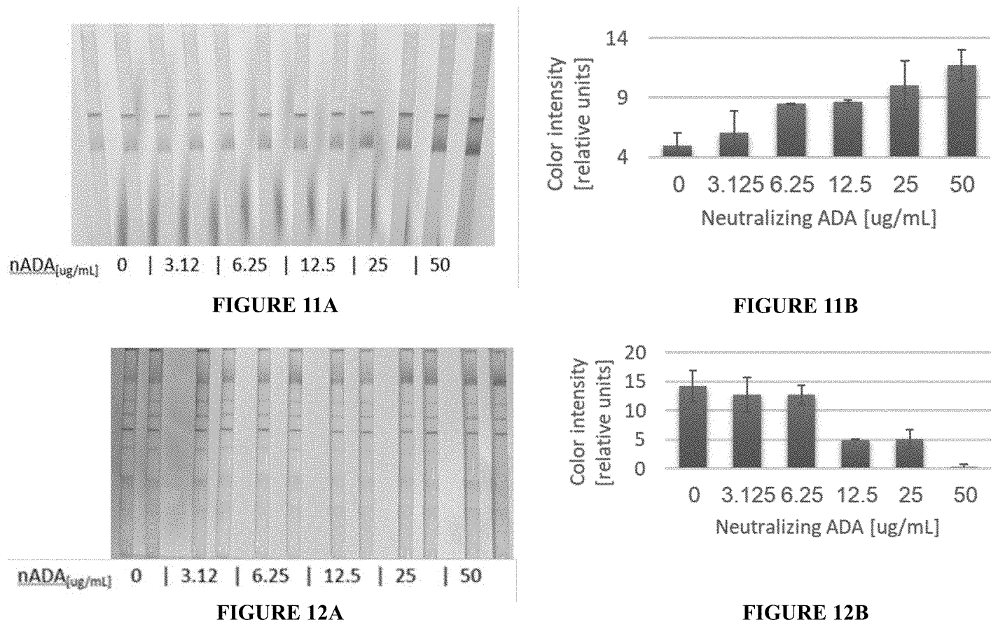

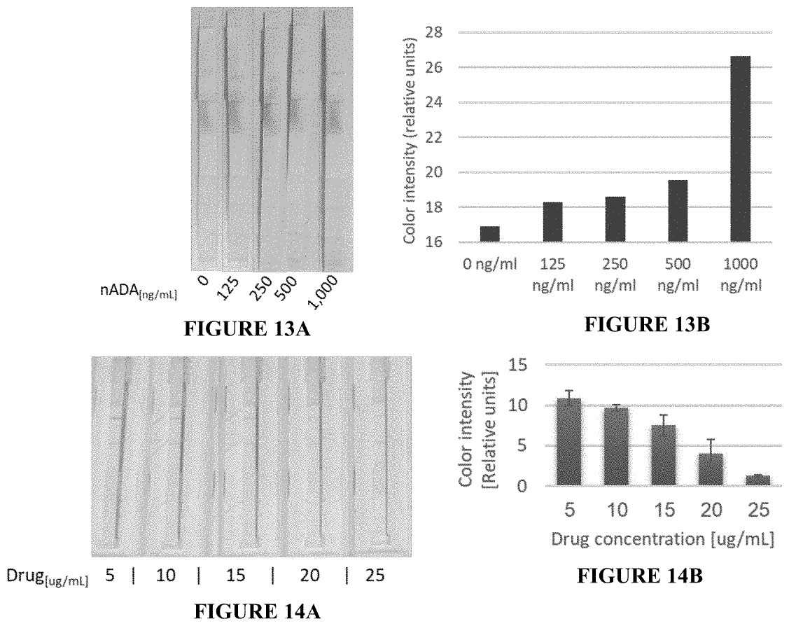

The inventors used the substrate line at the end side of the assay to quantify increasing concentrations of commercial neutralizing ADA with the same concentration of drug (50 μg/mL). As shown in FIG. 11A, there were visible differences between the different concentrations of neutralizing ADA. FIG. 11B, which is a graphical representation of FIG. 11A supports the visible results. After the sample has finished running on the strip, the inventors added DAB substrate to validate the results that were received from the substrate line. In FIG. 12A it can see observed that there were differences in the color intensity of the target molecule (such as TNFα; e.g., 182 in any one of FIGS. 9-10) capture line, which should capture the non-neutralized drug complexes. FIG. 12B which graphically represents these differences, shows that with growing concentrations of nADA the color intensity of the lowest capture line has decreased. Accordingly, the inventors concluded that there is a correlation between the color intensity of the substrate line and the color intensity of the target molecule (e.g., TNFα) line that represents the non-neutralized drug, which support the fact that the differences in the substrate line color intensity are related to the concentration of the neutralizing ADAs.

In this assay, the signal is derived from the complexes that were attached to neutralizing antibodies, and so they did not attach to the capture lines on the membrane in their migration.

### Example 3

### Capture Line Assay

The inventors dried anti-drug-Ab-HRP in concentration of 0.6 μg/mL in Tris saline 0.05 M with 0.5% BSA and 2% Lactose. The running buffer that was used was Tris saline 0.05 M with 1% of PEG 20K. As a sample, the inventors used spiked porcine serum. At a first experiment the inventors spiked the sample with different concentrations of drug, while in a second experiment the spiking was performed using increasing concentrations of commercial neutralizing ADAs. After the test was completed, the inventors added DAB substrate to the membrane and took pictures using mobile phone.

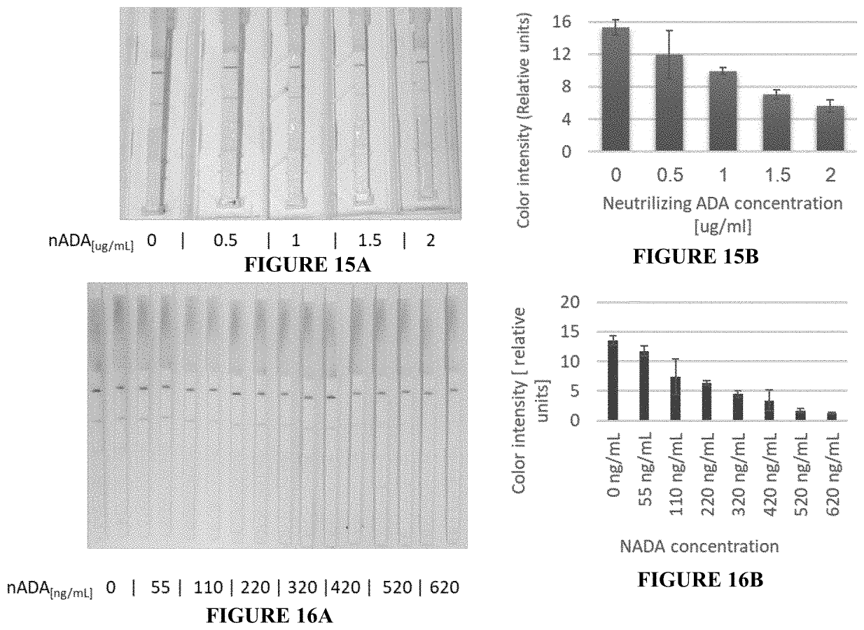

This assay measures the signal received from antibodies that are being attached on the capture lines. It has the capability of simultaneously detecting drug levels in the patient's serum as well as the neutralizing ADA's concentrations. On the one hand, FIG. 14 represents the dose response of the drug levels in serum. There is a decrease in the drug capture line's color intensity as the drug level increases, while on the other hand, with the increasing concentrations of nADA, the target molecule (e.g., TNFα) capture line's color intensity after adding the substrate is decreasing (FIG. 15). In FIG. 16 shown is a representation of the nADA dose response on the TNF line (first capture line), with LOD of 55 [ng/mL] with high resolution of 55-110 [ng/mL].

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

1. A device comprising a section 1, at least one section 2, at least one section 3, and a section 4, wherein:
a. said section 1 is coupled to said at least one section 2; and said section 3 is coupled to said section 2 and to said section 4;
b. said at least one section 3 comprises a surface functionalized with a target molecule and an agent having a specific binding affinity to said target molecule; and
c. sections 1 to 4 are: (i) arranged along a horizontal axis; and (ii) in liquid communication, allowing lateral flow of a liquid sequentially from said sections 1 to 4.

2. The device of claim 1, wherein:
a. said section 1 comprises a sample collecting surface; and
b. said at least one section 2 comprises a surface comprising said agent and an agent probing molecule having specific binding affinity to said agent.

3. The device of claim 1 or 2, wherein said at least one section 2 comprises two separate sections 2, wherein a first section 2 comprises a surface comprising said agent, and a second section 2 comprises a surface comprising said agent probing molecule having specific binding affinity to said agent, optionally wherein said probing molecule is linked to a reporter molecule, and wherein said reporter molecule generates a trigger.

4. (canceled)

5. The device of claim 1, wherein said at least one section 3 comprises two separate sections 3, wherein a first section 3 comprises a surface functionalized with a target molecule, and second section 3 comprises a surface functionalized with an agent having a specific binding affinity to said target molecule, optionally wherein said section 4 comprises a surface in contact with a substrate molecule generating a signal in response to said trigger.

6. (canceled)

7. The device of claim 1, further comprising a section 5, optionally wherein said section 5 comprises a surface in contact with a substrate molecule generating a signal in response to said trigger.

8. (canceled)

9. The device of claim 3, wherein said reporter molecule is selected from the group consisting of: an enzyme, a radioactive molecule, a luminescent compound, a fluorescent compound, a magnetic particle, an electro-chemiluminescent compound, a fluorescence transducing aptamer, and an electrochemically active compound, optionally wherein said trigger comprises: a reactive compound, electromagnetic radiation, a charged particle, or any combination thereof.

10. (canceled)

11. The device of claim 2, wherein said section 3 and said section 4 are devoid of said probing molecule and said reporter molecule, optionally wherein said probing molecule is an antibody.

12. (canceled)

13. The device of claim 1, wherein said target molecule comprises a peptide, optionally wherein said target molecule is selected from the group consisting of: a cytokine, a chemokine, an integrin, an adhesion molecule, and an immune checkpoint molecule.

14. (canceled)

15. The device of claim 1, wherein said agent is a drug affecting said target molecule, optionally wherein said agent comprises an antibody.

16. (canceled)

17. The device of claim 1, wherein said coupled is in contact or at least partially overlapping.

18. The device of claim 5, further comprising a detection unit in operable communication with said device, and wherein said detection unit is configured to detect said signal, optionally wherein said detection unit comprises an element selected form the group consisting of: an active-pixel sensor (APS), an electrode, an excitation source with active-pixel sensor, and any combination thereof.

19. (canceled)

20. A method for determining the presence of an analyte in a sample, comprising the steps of:
a. contacting section 1 of the device of claim 1 with a sample; and
b. detecting the presence of a signal,
wherein the presence of said signal is indicative of the presence of said analyte in said sample, thereby determining the presence of said analyte in the sample.

21. The method of claim 20, further comprising a step of quantifying the amount of said analyte in a sample, comprising: determining the amount of said signal, and comparing it to a calibration curve or an indicative value, thereby quantifying the amount of said analyte in the sample.

22. The method of claim 20, wherein said analyte comprises an antibody, optionally wherein said antibody comprises an antibody drug, a neutralizing antibody of said antibody drug, or both.

23. (canceled)

24. The method of claim 22, wherein said drug comprises an immune checkpoint inhibitor, optionally wherein said drug targets a cytokine.

25. (canceled)

26. The method of claim 22, further comprising a step of determining the amount of said drug in said sample.

27. The method of claim 20, wherein said sample is obtained or derived from a subject, optionally wherein said determining the presence of said analyte comprises determining the presence, the amount, or both, of an antibody drug, a neutralizing antibody of said antibody drug, or both in said sample or a subject.

28. (canceled)

29. The method of claim 27, wherein said subject is afflicted with a cell proliferation related disease, an immune disease, or both.

30. The method of claim 29, wherein said cell proliferation related disease comprises cancer.

31. The method of claim 29, wherein said immune disease comprises an autoimmune disease, an inflammatory disease, or both.
